(19) United States
(12) Patent Application Publication
(10) Pub. No.: US 2022/0202338 A1
(43) Pub. Date: Jun. 30, 2022

(54) SYSTEM FOR CREATING A COMPOSITE MAP

(71) Applicant: Acutus Medical, Inc., Carlsbad, CA (US)

(72) Inventors: Min ZHU, San Marcos, CA (US); Nathan ANGEL, Oceanside, CA (US); Pratik SHAH, Vista, CA (US); Xinwei SHI, San Diego, CA (US); Derrick CHOU, San Diego, CA (US); Graydon BEATTY, Carlsbad, CA (US); Maxwell R. FLAHERTY, Topsfield, MA (US)

(21) Appl. No.: 17/601,661

(22) PCT Filed: Apr. 17, 2020
(86) PCT No.: PCT/US2020/028779

§ 371 Date: Oct. 5, 2021

Related U.S. Application Data
(60) Provisional application No. 62/835,538, filed on Apr. 18, 2019.
(60) Provisional application No. 62/925,030, filed on Oct. 23, 2019.

(51) Int. Cl. A61B 5/333; A61B 18/14; A61B 5/287; A61B 5/339; A61B 5/352; A61B 5/367; G16H 50/50

(52) U.S. Cl. CPC A61B 5/333; A61B 18/1492; A61B 5/287; A61B 2017/00053; A61B 5/352; A61B 5/367; G16H 50/50; A61B 5/339

(57) ABSTRACT

Provided herein are systems for modeling a patients cardiac electrical activity data, including at least one diagnostic catheter for insertion into the heart of the patient and a processing unit. The at least one diagnostic catheter includes at least one recording element to record patient data over multiple cardiac cycles. The patient data includes biopotential data and localization data of the at least one recording element. The processing unit includes a clustering routine that: receives the recorded patient data; segments the recorded patient data by cardiac cycle to produce segmented patient data; groups the segments based on one or more characteristics of the segments to produce segmented data groups; and combines the segmented patient data within each segmented data group to produce one or more composite recordings. The systems create one or more models of cardiac electrical activity of the patient based on the one or more composite recordings.

### RELATED APPLICATIONS

The present application, while not claiming priority to, may be related to U.S. Provisional Application Ser. No. 62/835,538, entitled “System for Creating a Composite Map”, filed Apr. 18, 2019, which is hereby incorporated by reference.

The present application, while not claiming priority to, may be related to U.S. application Ser. No. 16/335,893, entitled “Ablation System with Force Control”, filed Mar. 220, 2019, which is a 35 USC 371 national stage filing of Patent Cooperation Treaty Application No. PCT/US2017/056064, entitled “Ablation System with Force Control”, filed Oct. 11, 2017, published as WO2018071490, which claims priority to U.S. Provisional Application Ser. No. 62/406,748, entitled “Ablation System with Force Control”, filed Oct. 11, 2016, and U.S. Provisional Application Ser. No. 62/504,139, entitled “Ablation System with Force Control”, filed May 20, 2017, each of which is hereby incorporated by reference.

The present application, while not claiming priority to, may be related to Patent Cooperation Treaty Application No. PCT/US2017/030915, entitled “Cardiac Information Dynamic Display System and Method”, filed May 3, 2017, which claims priority to U.S. Provisional Patent Application Ser. No. 62/331,351, entitled “Cardiac Information Dynamic Display System and Method”, filed May 3, 2016, each of which is hereby incorporated by reference.

The present application, while not claiming priority to, may be related to U.S. application Ser. No. 14/422,941, entitled “Catheter, System and Methods of Medical Uses of Same, Including Diagnostic and Treatment Uses for the Heart”, filed Feb. 20, 2015, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, published as WO 2014/036439, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. application Ser. No. 14/762,944, entitled “Expandable Catheter Assembly with Flexible Printed Circuit Board (PCB) Electrical Pathways”, filed Jul. 23, 2015, which is a 35 USC 371 national stage filing of Patent Cooperation Treaty Application No. PCT/US2014/015261, entitled “Expandable Catheter Assembly with Flexible Printed Circuit Board (PCB) Electrical Pathways”, filed Feb. 7, 2014, published as WO 2014/124231, which claims priority to U.S. Patent Provisional Application Ser. No. 61/762,363, entitled “Expandable Catheter Assembly with Flexible Printed Circuit Board (PCB) Electrical Pathways”, filed Feb. 8, 2013, each of which is hereby incorporated by reference.

The present application, while not claiming priority to, may be related to U.S. patent application Ser. No. 14/865,435, entitled “Method and Device for Determining and Presenting Surface Charge and Dipole Densities on Cardiac Walls”, filed Sep. 25, 2015, which is a continuation of U.S. Pat. No. 9,167,982, entitled “Method and Device for Determining and Presenting Surface Charge and Dipole Densities on Cardiac Walls”, filed Nov. 19, 2014, which is a continuation of U.S. Pat. No. 8,918,158 (hereinafter the '158 patent), entitled “Method and Device for Determining and Presenting Surface Charge and Dipole Densities on Cardiac Walls”, issued Dec. 23, 2014, which is a continuation of U.S. Pat. No. 8,700,119 (hereinafter the '119 patent), entitled “Method and Device for Determining and Presenting Surface Charge and Dipole Densities on Cardiac Walls”, issued Apr. 15, 2014, which is a continuation of U.S. Pat. No. 8,417,313 (hereinafter the '313 patent), entitled “Method and Device for Determining and Presenting Surface Charge and Dipole Densities on Cardiac Walls”, issued Apr. 9, 2013, which was a 35 USC 371 national stage filing of PCT Application No. CH2007/000380, entitled “Method and Device for Determining and Presenting Surface Charge and Dipole Densities on Cardiac Walls”, filed Aug. 3, 2007, published as WO 2008/014629, which claimed priority to Swiss Patent Application No. 1251/06 filed Aug. 3, 2006, each of which is hereby incorporated by reference.

The present application, while not claiming priority to, may be related to U.S. patent application Ser. No. 14/886,449, entitled “Device and Method for the Geometric Determination of Electrical Dipole Densities on the Cardiac Wall”, filed October 19, 2015, which is a continuation of U.S. Pat. No. 9,192,318, entitled “Device and Method for the Geometric Determination of Electrical Dipole Densities on the Cardiac Wall”, filed Jul. 19, 2013, which is a continuation of U.S. Pat. No. 8,512,255, entitled “Device and Method for the Geometric Determination of Electrical Dipole Densities on the Cardiac Wall”, issued Aug. 20, 2013, published as US2010/0298690 (hereinafter the '690 publication), which was a 35 USC 371 national stage application of Patent Cooperation Treaty Application No. PCT/IB09/00071 filed Jan. 16, 2009, entitled “A Device and Method for the Geometric Determination of Electrical Dipole Densities on the Cardiac Wall”, published as WO2009/090547, which claimed priority to Swiss Patent Application 00068/08 filed Jan. 17, 2008, each of which is hereby incorporated by reference.

The present application, while not claiming priority to, may be related to U.S. application Ser. No. 14/003,671, entitled “Device and Method for the Geometric Determination of Electrical Dipole Densities on the Cardiac Wall”, filed Sep. 6, 2013, which is a 35 USC 371 national stage filing of Patent Cooperation Treaty Application No. PCT/US2012/028593, entitled “Device and Method for the Geometric Determination of Electrical Dipole Densities on the Cardiac Wall”, published as WO2012/122517 (hereinafter the '517 publication), which claimed priority to U.S. Patent Provisional Application Ser. No. 61/451,357, each of which is hereby incorporated by reference.

The present application, while not claiming priority to, may be related to U.S. Design Application Ser. No. 29/475,273, entitled “Catheter System and Methods of Medical Uses of Same, Including Diagnostic and Treatment Uses for the Heart”, filed Dec. 2, 2013, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, which claims priority to U.S. Patent Provisional Application Ser. No. 61/695,535, entitled “System and Method for Diagnosing and Treating Heart Tissue”, filed Aug. 31, 2012, which is hereby incorporated by reference.

The present application, while not claiming priority to, may be related to Patent Cooperation Treaty Application No. PCT/US2014/15261, entitled “Expandable Catheter Assembly with Flexible Printed Circuit Board (PCB) Electrical Pathways”, filed Feb. 7, 2014, which claims priority to U.S. Patent Provisional Application Ser. No. 61/762,363, entitled “Expandable Catheter Assembly with Flexible Printed Circuit Board (PCB) Electrical Pathways”, filed Feb. 8, 2013, which is hereby incorporated by reference.

The present application, while not claiming priority to, may be related to Patent Cooperation Treaty Application No. PCT/US2015/11312, entitled “Gas-Elimination Patient Access Device”, filed Jan. 14, 2015, which claims priority to U.S. Patent Provisional Application Ser. No. 61/928,704, entitled “Gas-Elimination Patient Access Device”, filed Jan. 17, 2014, which is hereby incorporated by reference.

The present application, while not claiming priority to, may be related to Patent Cooperation Treaty Application No. PCT/US2015/22187, entitled “Cardiac Analysis User Interface System and Method”, filed March 24, 2015, which claims priority to U.S. Patent Provisional Application Ser. No. 61/970,027, entitled “Cardiac Analysis User Interface System and Method”, filed Mar. 28, 2014, which is hereby incorporated by reference.

The present application, while not claiming priority to, may be related to Patent Cooperation Treaty Application No. PCT/US2014/54942, entitled “Devices and Methods for Determination of Electrical Dipole Densities on a Cardiac Surface”, filed Sep. 10, 2014, which claims priority to U.S. Patent Provisional Application Ser. No. 61/877,617, entitled “Devices and Methods for Determination of Electrical Dipole Densities on a Cardiac Surface”, filed Sep. 13, 2013, which is hereby incorporated by reference.

The present application, while not claiming priority to, may be related to U.S. Patent Provisional Application Ser. No. 62/161,213, entitled “Localization System and Method Useful in the Acquisition and Analysis of Cardiac Information”, filed May 13, 2015, which is hereby incorporated by reference.

The present application, while not claiming priority to, may be related to U.S. Patent Provisional Application Ser. No. 62/160,501, entitled “Cardiac Virtualization Test Tank and Testing System and Method”, filed May 12, 2015, which is hereby incorporated by reference.

The present application, while not claiming priority to, may be related to U.S. Patent Provisional Application Ser. No. 62/160,529, entitled “Ultrasound Sequencing System and Method”, filed May 12, 2015, which is hereby incorporated by reference.

The present application, while not claiming priority to, may be related to U.S. Patent Provisional Application Ser. No. 62/619,897, entitled “System for Recognizing Cardiac Conduction Patterns”, filed Jan. 21, 2018, which is hereby incorporated by reference.

The present application, while not claiming priority to, may be related to U.S. Patent Provisional Application Ser. No. 62/668,647, entitled “System for Identifying Cardiac Conduction Patterns”, filed May 8, 2018, which is hereby incorporated by reference.

The present application, while not claiming priority to, may be related to U.S. Provisional Patent Application Ser. No. 62/668,659, entitled “Cardiac Information Processing System”, filed May 8, 2018, incorporated herein by reference.

The present application, while not claiming priority to, may be related to U.S. Provisional Patent Application Ser. No. 62/757,961, entitled “Systems and Methods for Calculating Patient Information”, filed Nov. 9, 2018, incorporated herein by reference.

The present application, while not claiming priority to, may be related to U.S. Provisional Patent Application Ser. No. 62/811,735, entitled “Cardiac Information Processing System”, filed Feb. 28, 2019, incorporated herein by reference.

### FIELD OF THE INVENTION

The present invention relates generally to systems and methods for the diagnosis and treatment of cardiac arrhythmias or other abnormalities, in particular, the present invention is related to systems, devices, and methods for mapping cardiac electrical activity.

### BACKGROUND

Cardiac signals (e.g., charge density, dipole density, voltage, etc.) vary across the endocardial surface in magnitude. The magnitude of these signals is dependent on several factors, including local tissue characteristics (e.g., healthy vs. disease/scar/fibrosis/lesion) and regional activation characteristics (e.g., “electrical mass” of activated tissue prior to activation of the local cells). A common practice is to assign a single threshold for all signals at all times across the surface. The use of a single threshold can cause low-amplitude activation to be missed or cause high-amplitude activation to dominate/saturate, leading to confusion in interpretation of the map. Failure to properly detect activation can lead to imprecise identification of regions of interest for therapy delivery or incomplete characterization of ablation efficacy (excess or lack of block).

The continuous, global mapping of atrial fibrillation yields a tremendous volume of temporally-variable and spatially-variable activation patterns. A limited, discrete sampling of map data may be insufficient to provide a comprehensive picture of the drivers, mechanisms, and supporting substrate for the arrhythmia. Clinician review of long durations of AF can be challenging to remember and create a valuable clinical diagnosis. There is a need for improved systems, methods, and devices for mapping cardiac electrical activity.

### SUMMARY

According to one aspect of the present inventive concepts, a system for modeling a patient's cardiac electrical activity data, comprising: at least one diagnostic catheter for insertion into the heart of the patient, the at least one diagnostic catheter comprising at least one recording element configured to record patient data over multiple cardiac cycles, the patient data comprising: biopotential data; and localization data comprising the location of the at least one recording element; and a processing unit comprising a clustering routine configured to: receive the recorded patient data; segment the recorded patient data by cardiac cycle to produce segmented patient data comprising segments; group the segments based on one or more characteristics of the segments to produce segmented data groups; and combine the segmented patient data within each segmented data group to produce one or more composite recordings. The system can be configured to create one or more models of cardiac electrical activity of the patient based on the one or more composite recordings.

In some embodiments, the one or more models of cardiac electrical activity comprise two or more models of cardiac electrical activity.

In some embodiments, the biopotential data comprises biopotential signals recorded by each of the at least one recording elements, and segmenting the recorded patient data comprises segmenting each of the biopotential signals by cardiac cycle into multiple biopotential signal segments; and each of the one or more composite recordings comprises two or more of the multiple biopotential signal segments. The two or more of the multiple biopotential signal segments can comprise at least 1,000 biopotential signal segments. The two or more of the multiple biopotential signal segments can comprise at least 2,000 biopotential signal segments. The two or more of the multiple biopotential signal segments can comprise at least 5,000 biopotential signal segments.

In some embodiments, the one or more segment characteristics are selected from the group consisting of: pattern; cycle length; signal morphology; amplitude; frequency; frequency components; wavelet composition; and combinations thereof.

In some embodiments, the clustering routine comprises an algorithm selected from the group consisting of: a connectivity model-based algorithm, such as a hierarchical clustering algorithm, and the models are based on distance connectivity; a centroid model-based algorithm, such as a k-means clustering algorithm, and each cluster is represented by a single mean vector; a density model-based algorithm that defines the clusters as connected dense regions in the data space; a distribution model-based algorithm, such as a Gaussian mixture model clustering algorithm, and the clusters are modeled using a statistical distribution, such as a multivariate normal distribution; a graph-based model algorithm; a neural model-based algorithm, such as a self-organizing map and/or other unsupervised neural network, and an artificial neural network and/or other non-linear statistical data modeling tool is used to model complex relationships and patterns in data; and combinations thereof.

In some embodiments, the system further comprises an automatic timing annotation algorithm configured to identify and annotate one or more signal characteristics of the cardiac electrical activity. The characteristics can correspond to cardiac tissue depolarization, activation, and/or repolarization.

In some embodiments, the segments are grouped based on template matching. The template matching can be based on one or more segment templates. The one or more segment templates can be dynamically adjusted.

In some embodiments, one or more of the segmented data groups are merged to form a merged group of all segments within the one or more segmented data groups.

In some embodiments, the system further comprises a display. The one or more models of cardiac electrical activity can be shown on the display. The one or more models can be shown on the display during the recording. A portion of the one or more models can be shown on the display during the recording. An operator can be shown visual feedback information on the display in a closed loop fashion. The feedback information can be configured to provoke the operator to perform an action selected from the group consisting of: expand, extend, and/or alter an operator-determined pattern to include regions with lack of data, insufficient data, and/or poorly spatially distributed data; to increase data quantity and/or quality in a specific region of interest; to replace data in a region; to achieve full coverage, including high quality data across the full cardiac chamber surface and/or throughout the whole volume being assessed; and combinations thereof. The visual feedback information can be configured to indicate the quantity and/or the quality of the recorded patient data. The quantity and/or quality can be determined over time. The quantity and/or quality can be determined across space.

In some embodiments, at least one of the at least one recording elements is in contact with the cardiac tissue for at least a portion of the data recording.

In some embodiments, at least one of the at least one recording elements is not in contact with the cardiac tissue for at least a portion of the data recording. At least one of the at least one recording elements can be in contact with the cardiac tissue for the at least a portion of the data recording. Data recorded by recoding elements in contact with cardiac tissue can be processed separately from data recorded by recording elements not in contact with tissue.

In some embodiments, the at least one diagnostic catheter comprises at least two diagnostic catheters, and each diagnostic catheter comprises at least one recording element.

In some embodiments, the at least one recording element comprises an array of recording elements. The array can comprise a basket array. The array can comprise at least 48 recording elements. The array of recording elements can be maneuvered during a recording to cover at least 25%, 40%, and/or 60% of the volume of the cardiac chamber.

In some embodiments, the clustering routine comprises a detect-reject algorithm configured to identify undesirable signal characteristics.

In some embodiments, the clustering routine is configured to filter inconsistent, erroneous, and/or otherwise unwanted data.

The technology described herein, along with the attributes and attendant advantages thereof, will best be appreciated and understood in view of the following detailed description taken in conjunction with the accompanying drawings in which representative embodiments are described by way of example.

In accordance with an aspect of the inventive concepts, provided is a system for modeling cardiac electrical activity data, comprising: a plurality of transducers to sense the positions of features of the patient's heart; a plurality of sensors to sense biopotential data of the patient's heart; a recording unit to record the sensed position and biopotential data; and a processing unit comprising a clustering routine. The processing unit includes one or more processors configured to: receive recorded patient data, including sensed position and biopotential data related to the patient's heart; segment the recorded patient data by cardiac cycle to produce segmented patient data comprising segments; group the segments based on one or more characteristics of the segments to produce segmented data groups; and combine the segmented patient data within each segmented data group to produce one or more composite recordings. The system is configured to create one or more models of cardiac electrical activity of the patient based on the one or more composite recordings.

In various embodiments, the biopotential data comprises biopotential signals produced by each of the sensors, wherein segmenting the recorded patient data comprises segmenting each of the biopotential signals by cardiac cycle into multiple biopotential signal segments; and/or wherein each of the one or more composite recordings comprises two or more of the multiple biopotential signal segments.

In various embodiments, the one or more segment characteristics are selected from the group consisting of: pattern; cycle length; signal morphology; amplitude; frequency; frequency components; wavelet composition; and combinations thereof.

In various embodiments, the clustering routine comprises an algorithm selected from the group consisting of: a connectivity model-based algorithm, such as a hierarchical clustering algorithm, wherein the models are based on distance connectivity; a centroid model-based algorithm, such as a k-means clustering algorithm, wherein each cluster is represented by a single mean vector; a density model-based algorithm that defines the clusters as connected dense regions in the data space; a distribution model-based algorithm, such as a Gaussian mixture model clustering algorithm, wherein the clusters are modeled using a statistical distribution, such as a multivariate normal distribution; a graph-based model algorithm; a neural model-based algorithm, such as a self-organizing map and/or other unsupervised neural network, wherein an artificial neural network and/or other non-linear statistical data modeling tool is used to model complex relationships and patterns in data; and combinations thereof.

In various embodiments, the system further comprises a display, wherein the one or more models are shown on the display during the recording and an operator is shown visual feedback information on the display in a closed loop fashion.

In various embodiments, the feedback information is configured to provoke the operator to perform an action selected from the group consisting of: expand, extend, and/or alter an operator-determined pattern to include regions with lack of data, insufficient data, and/or poorly spatially distributed data; to increase data quantity and/or quality in a specific region of interest; to replace data in a region; to achieve full coverage, including high quality data across the full cardiac chamber surface and/or throughout the whole volume being assessed; and combinations thereof.

In various embodiments, the clustering routine comprises a detect-reject algorithm configured to identify undesirable signal characteristics.

In various embodiments, the clustering routine is configured to filter inconsistent, erroneous, and/or otherwise unwanted data.

In accordance with another aspect of the inventive concepts, provided is an ablation system, comprising: at least one diagnostic catheter for insertion into the heart of the patient, the at least one diagnostic catheter comprising at least one recording element configured to record patient data over multiple cardiac cycles. The patient data comprises biopotential data and localization data comprising the location of the at least one recording element. The system also includes an ablation catheter comprising an elongate shaft with a distal portion and at least one ablation element positioned on the ablation catheter shaft distal portion and configured to deliver energy to tissue. And the system includes a processing unit comprising a clustering routine configured to: receive the recorded patient data; segment the recorded patient data by cardiac cycle to produce segmented patient data comprising segments; group the segments based on one or more characteristics of the segments to produce segmented data groups; and combine the segmented patient data within each segmented data group to produce one or more composite recordings. The system is configured to create one or more models of cardiac electrical activity of the patient based on the one or more composite recordings.

In various embodiments, the biopotential data comprises biopotential signals produced by each of the sensors, wherein segmenting the recorded patient data comprises segmenting each of the biopotential signals by cardiac cycle into multiple biopotential signal segments; and/or wherein each of the one or more composite recordings comprises two or more of the multiple biopotential signal segments.

In various embodiments, the one or more segment characteristics are selected from the group consisting of: pattern; cycle length; signal morphology; amplitude; frequency; frequency components; wavelet composition; and combinations thereof.

In various embodiments, the clustering routine comprises an algorithm selected from the group consisting of: a connectivity model-based algorithm, such as a hierarchical clustering algorithm, wherein the models are based on distance connectivity; a centroid model-based algorithm, such as a k-means clustering algorithm, wherein each cluster is represented by a single mean vector; a density model-based algorithm that defines the clusters as connected dense regions in the data space; a distribution model-based algorithm, such as a Gaussian mixture model clustering algorithm, wherein the clusters are modeled using a statistical distribution, such as a multivariate normal distribution; a graph-based model algorithm; a neural model-based algorithm, such as a self-organizing map and/or other unsupervised neural network, wherein an artificial neural network and/or other non-linear statistical data modeling tool is used to model complex relationships and patterns in data; and combinations thereof.

In various embodiments, the system further comprises a display, wherein the one or more models are shown on the display during the recording and an operator is shown visual feedback information on the display in a closed loop fashion.

In various embodiments, the feedback information is configured to provoke the operator to perform an action selected from the group consisting of: expand, extend, and/or alter an operator-determined pattern to include regions with lack of data, insufficient data, and/or poorly spatially distributed data; to increase data quantity and/or quality in a specific region of interest; to replace data in a region; to achieve full coverage, including high quality data across the full cardiac chamber surface and/or throughout the whole volume being assessed; and combinations thereof.

In various embodiments, the clustering routine comprises a detect-reject algorithm configured to identify undesirable signal characteristics.

In various embodiments, the clustering routine is configured to filter inconsistent, erroneous, and/or otherwise unwanted data.

In various embodiments, the system further comprises an energy source configured to provide energy to the at least one ablation element of the ablation catheter, wherein the energy source is configured to provide an energy form selected from the group consisting of: radiofrequency energy; cryogenic energy; laser energy; light energy; microwave energy; ultrasound energy; chemical energy; and combinations thereof.

In accordance with another aspect of the inventive concepts, provided is an ablation system, comprising at least one diagnostic catheter for insertion into the heart of the patient, the at least one diagnostic catheter comprising at least one recording element configured to record patient data over multiple cardiac cycles. the patient data comprises biopotential data and localization data comprising the location of the at least one recording element. The system also includes an ablation catheter comprising: an elongate shaft with a distal portion and at least one ablation element positioned on the ablation catheter shaft distal portion and configured to deliver energy to tissue. The system also comprises a processing unit comprising a clustering routine configured to: receive the recorded patient data; segment the recorded patient data by cardiac cycle to produce segmented patient data comprising segments; group the segments based on one or more characteristics of the segments to produce segmented data groups; and combine the segmented patient data within each segmented data group to produce one or more composite recordings. The system is configured to create one or more models of cardiac electrical activity of the patient based on the one or more composite recordings. The system also includes a display, wherein at least a portion of one of the one or more models of cardiac electrical activity are shown on the display.

In various embodiments, the system is configured to generate one or more active area plots on the display.

In various embodiments, the system is configured to generate one or more streamline plots on the display.

In various embodiments, the system is configured to generate one or more auto path plots on the display.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a schematic view of a system configured to perform a cardiac mapping procedure, consistent with the present inventive concepts.

FIG. 2 illustrates a flowchart of a method for recording and modeling electrical activity of a patient, consistent with the present inventive concepts.

FIG. 3A, 3C, and 3D illustrate graphs of recorded electrical activity, consistent with the present inventive concepts.

FIG. 3B illustrates a graph of time-aligned recorded electrical activity, consistent with the present inventive concepts.

FIG. 4 illustrates a visual representation of pattern clustering, consistent with the present inventive concepts.

FIG. 5A, 5B, 5C, and 5D illustrate a graphical representation of a cardiac chamber with multiple recording locations, a graph of multiple cardiac electrical activity recordings, and an activation map, respectively, consistent with the present inventive concepts.

FIG. 6 illustrates a flowchart of a method for recording and modeling electrical activity of a patient, consistent with the present inventive concepts.

FIG. 7 illustrates a flowchart of a method for recording and modeling electrical activity of a patient, consistent with the present inventive concepts.

FIGS. 8A-C provide various displays of cardiac activity maps, consistent with the present inventive concepts;

FIGS. 9A-D provide displays of sequential data acquisition, cluster samples, fuzzy membership functions, and bio data for beats within a given group, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems, methods, and device for modeling a patient's cardiac activity. The system includes at least one diagnostic catheter for insertion into the heart of a patient, the catheter including one or more recording elements (e.g. electrodes and/or ultrasound transducers) configured to record data (e.g. patient electrical data, patient anatomical data, and/or device location data) over multiple cardiac cycles. The system includes a processing unit comprising a clustering routine configured to: receive the recorded patient data; segment the recorded patient data by cardiac cycle to produce segmented patient data comprising segments; group the segments based on one or more characteristics of the segments to produce segmented data groups; and combine the segmented patient data within each segmented data group to produce one or more composite recordings. The system creates one or more models of cardiac electrical activity of the patient based on the one or more composite recordings. In some embodiments, multiple models of cardiac electrical activity are produced.

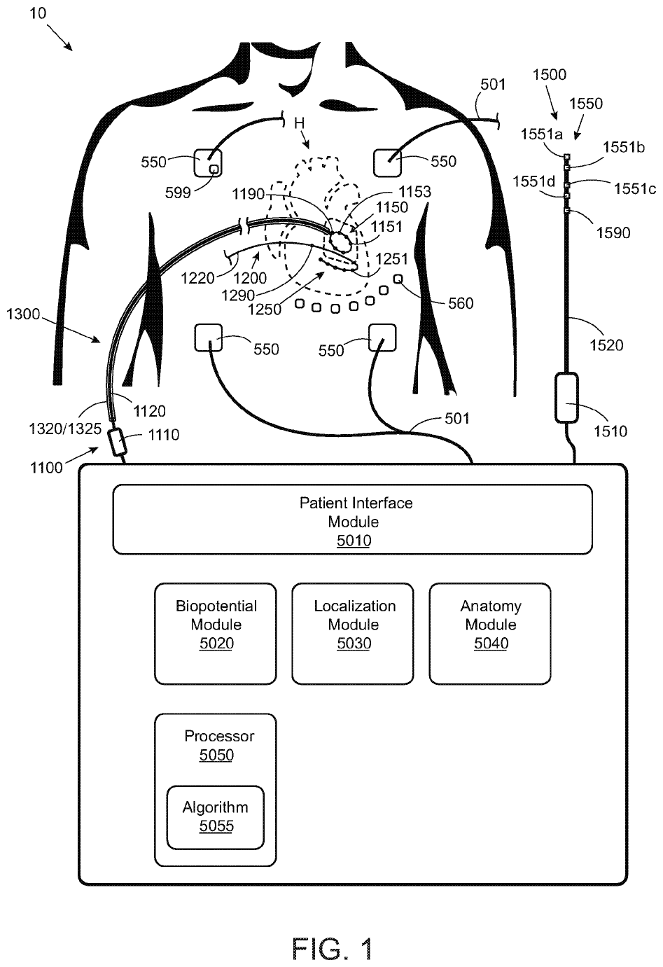

Referring now to FIG. 1, a schematic view of a system configured to perform a cardiac mapping procedure is illustrated, consistent with the present inventive concepts. System 10 can comprise a variety of components, subsystems, and the like, that are configured to cooperatively record and analyze physiological information, diagnose physiological conditions and/or maladies, and/or treat physiological conditions and/or maladies. System 10 can include console 5000 and one or more catheters 1000 for insertion into a patient.

Catheters 1000 can include one or more mapping catheters, mapping catheter 1100. Mapping catheter 1100 can include an array of one or more elements, basket array 1150. Basket array 1150 can comprise a compressible and/or expandable structure that includes these elements (e.g. electrodes and/or ultrasound transducers). Basket array 1150 can comprise: one or more splines; a linear array of elements; a circular or spiral array of elements; a grid of elements; and/or a multi-arm array of elements. Basket array 1150 can comprise a plurality of splines. One or more of the plurality of splines can include one or more functional elements (e.g. electrodes) configured to sense and/or record voltage potentials (also referred to as “potentials” herein) relating to cardiac activity and/or functional elements (e.g. electrodes and/or ultrasound transducers) used for localization. Array 1150 can include between three to eight splines, such as six splines, each comprising a plurality of sensing, recording, and/or localizing functional elements. Functional elements of basket 1150 can include electrodes 1151 and/or ultrasound transducers (USTs) 1153. Electrodes 1151 can be used for mapping, localization, and/or, in some embodiments, for delivering ablation energy. The electrodes 1151 can be coupled to console 5000, which can be configured to drive the electrodes 1151 and receive and record data from the electrodes 1151. Ultrasound transducers 1153 can include at least one ultrasound emitter and ultrasound sensor. The ultrasound transducers 1153 can be configured for localization of the basket array 1150 and/or localization of other catheters and/or structures within the heart H. Ultrasound transducers 1153 can also be configured for gathering data useful for generating and/or updating an image of the heart H and/or other anatomy of a patient. Catheter 1100 includes a catheter shaft 1120. Shaft 1120 can be configured to slide within a lumen 1325 of a shaft 1320 of a transseptal sheath or other introducer device, sheath 1300, used for insertion and translation inside the patient P, e.g., to deliver the basket array 1150 to the heart H.

A handle 1110 used to steer the catheter within the patient P is located at a proximal end of the catheter shaft 1120. The basket array 1150 extends from a distal end of the catheter shaft 1120. In various embodiments, the array 1150 can be or at least include an expandable/collapsible basket array coupled to a distal end of the catheter shaft 1120. An actuator (not shown but typically a flexible mechanical linkage) can be slidable within the shaft 1120 and it can be coupled to and/or engaged with a distal end of the array 1150. In various embodiments, the actuator extends distally to collapse array 1150, e.g., by straightening the splines 1157, and retracts proximally to expand array 1150, by outwardly bowing the splines 1157. The catheter 1100 can also comprise at least one other functional element 1190, such as an electrode, and/or other physiological sensor, located on catheter shaft 1120.

Catheters 1000 can include one or more diagnostic catheters, diagnostic catheter 1200. Catheter 1200 can include a shaft 1220 for insertion into the patient P and delivery to the heart H. For example, in some embodiments, catheter 1200 can be a coronary sinus mapping catheter, which is structured and arranged for positioning within the coronary sinus of the heart H. Catheter 1200 can include an electrode array 1250 comprising one or more functional elements in the form of electrodes 1251, e.g., such as electrodes used in cardiac activity mapping and/or localization. The catheter 1200 can also comprise at least one other functional element 1290, such as an electrode, and/or other physiological sensor, which can be located on catheter shaft 1220.

Catheters 1000 can include one or more treatment catheters, treatment catheter 1500. The treatment catheter 1500 can be, as examples, an ablation catheter, such as radio frequency (RF) ablation catheter, an alternative light energy delivery catheter, a cryoablation catheter, an electric-field generating catheter (such as a pulsed electric field catheter), and/or an ultrasound or other sound energy catheter. The catheter 1500 can comprise a shaft 1520 with a handle 1510 at its proximal end. At a distal end of shaft 1520 is disposed a treatment array 1550 comprising at least one functional element 1551. As examples, the functional elements of the treatment array 1550 can include one or more type of energy delivery element(s) 1551, such as one or more RF delivery electrode, one or more optical component for delivering light energy, cold energy, and/or one or more sound transducer for delivering ultrasound energy. In some embodiments, electrodes 1551a-d can be used for ablation treatment, but in another embodiment, one electrode (1551a) could be used for ablation and one or more of the remaining electrodes 1551b-c could still exist for localization, e.g., if the array 1550 comprises a cryoablation tip (1551d). In various embodiments, functional element 1551a could be a treatment element (e.g., RF, CRYO, etc.), and functional elements 1551b,c,d could be electrodes for localizing treatment element 1551a. In another embodiment, the array 1550 includes four electrodes 1551a-d for RF ablation. The catheter 1500 can also comprise at least one other functional element 1590, such as an electrode, coil, and/or physiological sensor, located on catheter shaft 1520.

System 10 can include one or more patches 550. Patches 500 can comprise electrodes, magnetic elements, and/or combinations thereof. The patches 550 can be external to the patient, e.g., the patches 550 could be configured for application to a torso of the patient P. The patches 550 and catheters 1000, and/or components thereof, can be configured to provide data and information to the console 5000 to perform localization of one or more devices of system 10 within and/or on patient P. Localization can be performed as described in applicants co-pending U.S. patent application Ser. No. 15/569,457, titled “LOCALIZATION SYSTEM AND METHOD USEFUL IN THE ACQUISITION AND ANALYSIS OF CARDIAC INFORMATION”, filed Oct. 26, 2017, the content of which is incorporated herein in its entirety for all purposes.

The patches 550 can be coupled to the console 5000 with one or more cables and/or cable assemblies 501, and/or other wired or wireless data transfer element. The patches 550 can be skin-contacting patches including an adhesive for removable application to the torso of the patient P, and each patch can include one or more types of functional elements. The patches 550 can include at least one impedance functional element, such as an electrode, configured to measure impedance at the patch and/or provide a drive signal for an impedance-based localization modality. The impedance measurements can be used by console 5000 to perform impedance-based localization.

In some embodiments, one or more of the patches 550 can be a combination (or “combo”) patch including two or more different types of functional elements. As examples, a combo patch 550 can include at least one magnetic element and at least one impedance element, and it can optionally include another type of functional element 599. For example, functional element can be a 12 lead EKG/ECG (electrocardiogram) element generally positioned on a patient during a clinical procedure. Alternatively or additionally, system 10 can include one or more EKG/ECG electrodes (e.g. electrode patches) 560.

In various embodiments, any one or more of patches 550 can include at least one other functional element 599. Beyond an EKG/ECG-based functional element, the functional element 599 can be or at least include, as examples, a generic sensor, transducer, and/or other functional element, e.g. accelerometer, sweat detector, physiologic sensor, and/or imaging marker (e.g. radiopaque marker, MR marker). As other examples, in some embodiments, functional element 599 comprises a microwave functional element, an ultrasound functional element, or a combination thereof.

Console 5000 can include a patient interface module 5010, a biopotential module 5020, a localization module 5030, and an anatomy module 5040. Console 5000 can further comprise a processor 5050 and algorithm 5055. Patient interface module 5010 can be configured to operably attach one or more patient devices (e.g. one or more catheters 1000 and/or patches 550) to one or more components of console 5000. Patient interface module 5010 can provide patient electrical isolation, such that patient P is protected from any potentially harmful voltage and/or current sources within console 5000.

Biopotential module 5020, localization module 5030, and anatomy module 5040 can each be configured to receive data from a plurality of different external functional elements (e.g. functional elements of one or more catheters 1000), process the received data, and generate outputs, such as generated information shown on one or more displays of system 10 (displays not shown but typically one or more touch screens and/or other video displays), based at least on part on the processed data. Biopotential module 5020 can generate one or more outputs related to the electrical activity of the patient, for example dipole density information, surface charge information, and/or voltage information related to the activity of the patient's heart. Biopotential module can be of similar construction and arrangement to similar components described in applicants co-pending: U.S. patent application Ser. No. 16/014,370, titled “METHOD AND DEVICE FOR DETERMINING AND PRESENTING SURFACE CHARGE AND DIPOLE DENSITIES ON CARDIAC WALLS”, filed Jun. 21, 2018; U.S. patent application Ser. No. 15/882,097, titled “DEVICE AND METHOD FOR THE GEOMETRIC DETERMINATION OF ELECTRICAL DIPOLE DENSITIES ON THE CARDIAC WALL”, filed Jan. 29, 2018; U.S. patent application Ser. No. 29/681,827, titled “SET OF TRANSDUCER-ELECTRODE PAIRS FOR A CATHETER”, filed Feb. 28, 2019; U.S. patent application Ser. No. 16/097,959, titled “CARDIAC MAPPING SYSTEM WITH EFFICIENCY ALGORITHM”, filed Oct. 31, 2018; the content of each of which is incorporated herein in its entirety for all purposes. Localization module 5030 can generate one or more outputs related to the position of one or more components of system 10 relative to patient P, such as relative to a coordinate system established by localization module 5030. Localization module 5030 can be of similar construction and arrangement to similar components described in applicants co-pending U.S. patent application Ser. No. 15/569,457, titled “LOCALIZATION SYSTEM AND METHOD USEFUL IN THE ACQUISITION AND ANALYSIS OF CARDIAC INFORMATION”, filed Oct. 26, 2017, the content of which is incorporated herein in its entirety for all purposes. Anatomy module 5040 can generate one or more outputs related to the anatomy of patient P, for example the size, shape, and/or structure of at least a portion (e.g. a chamber) of heart H of patient P. Anatomy module 5040 can be of similar construction and arrangement to similar components described in applicants co-pending: U.S. patent application Ser. No. 29/681,827, titled “SET OF TRANSDUCER-ELECTRODE PAIRS FOR A CATHETER”, filed Feb. 28, 2019; U.S. patent application Ser. No. 15/569,185, titled “ULTRASOUND SEQUENCING SYSTEM AND METHOD”, filed Oct. 25, 2017; the content of each of which is incorporated herein in its entirety for all purposes.

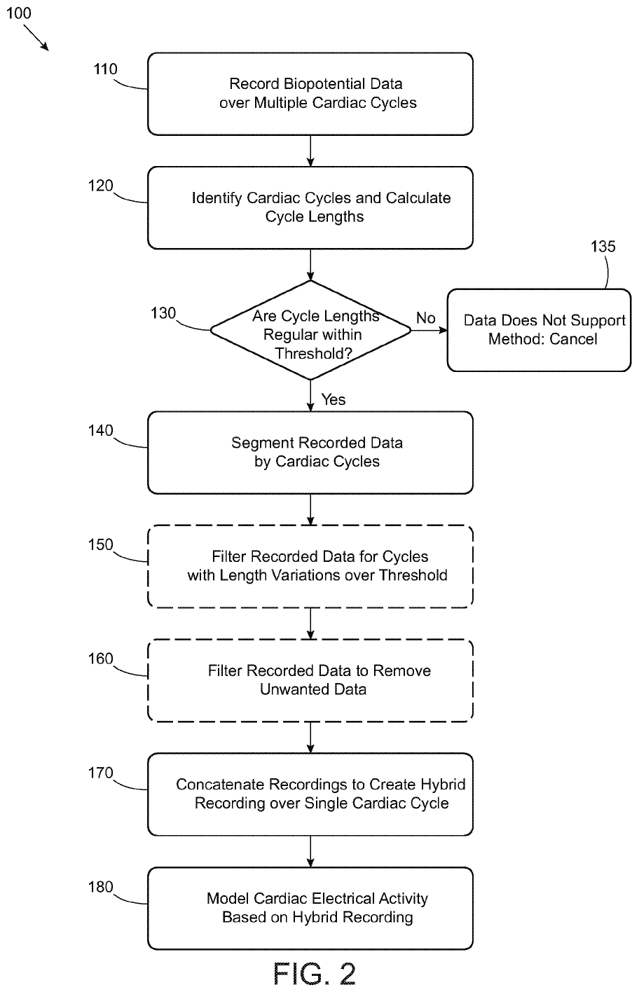

In some embodiments, biopotential module 5020, localization module 5030, and/or anatomy module 5040 produce one or more drive signals, such as one or more drive signals configured to drive one or more external functional elements of system 10 (e.g. localization electrodes 550 and/or ultrasound transducers 1153). In some embodiments, processor 5050 is configured to function cooperatively with biopotential module 5020, localization module 5030, and/or anatomy module 5040, such as to receive and analyze data, and generate one or more outputs. In some embodiments, outputs generated by biopotential module 5020, localization module 5030, and/or anatomy module 5040 are received as data inputs to processor 5050 and/or other modules of console 5000. In some embodiments, algorithm 5055 contains instructions configured to allow processor 5050 to perform one or more operations. For example, algorithm 5055 can comprise instructions to perform method 100 described herebelow in reference to FIG. 2.

Referring now to FIG. 2, a flowchart of a method for recording and modeling electrical activity of a patient is illustrated, consistent with the present inventive concepts.

Method 100 of FIG. 2 is described using various components of system 10 of FIG. 1 as described hereabove.

In Step 110, data is recorded (e.g. by console 5000) from recording elements (e.g. electrodes and/or ultrasound transducers) of one or more catheters 1000 inserted into a patient P. The data can be recorded over multiple cardiac cycles. In some embodiments, data is recorded when the recording elements are in contact with the cardiac tissue, not in contact with the cardiac tissue, or a combination of the two. In some embodiments, only data recorded from elements in contact with the cardiac tissue is processed. In some embodiments, only data recorded from elements not in contact with the cardiac tissue is processed. In some embodiments, data recorded by recording elements in contact with the cardiac tissue is processed together with data recorded by recording elements not in contact with tissue, for example in a composite (e.g. integrated) solution. In some embodiments, only data recorded by recording elements in contact with the cardiac tissue is processed (e.g. independently). In some embodiments, data recorded from elements in contact with the cardiac tissue is processed separately from data recorded from elements not in contact with the cardiac tissue. In some embodiments, cardiac information resulting from data processed separately based on the state of contact of the recording elements can be further processed in combination to improve the cardiac information. In some embodiments, cardiac information resulting from data processed separately based on the state of contact of the recording elements can be quantitatively compared and results of the quantitative comparison can be displayed. In some embodiments, the results of the quantitative comparison of cardiac information at various locations on the heart anatomy can be displayed on a two- or three-dimensional representation (such as an image) of the heart anatomy.

In some embodiments, data is recorded from a catheter including an array of recording elements, such as mapping catheter 1100. As described hereabove, the array of recording elements can be positioned on a basket and/or other radially expandable structure. In some embodiments, the array of recording elements is positioned linearly along a shaft, circularly or in a spiral along a shaft, in a grid configuration, and/or in a multi-arm catheter where each arm is attached to the shaft on one end (e.g. similar to the geometry of a household mop). Data can also be recorded from a reference catheter, such as coronary sinus catheter 1200. For example, the distal portion of catheter 1200 (e.g. at least electrode array 1250) can be positioned within the coronary sinus of the heart H of patient P. Electrode array 1150 (e.g. a basket array of at least electrodes 1151) of catheter 1100 is positioned within a chamber of heart H, such as within the left atrium of heart H. Data (e.g. biopotential data and/or localization data) is recorded from both catheters 1100 and 1200. As the data is recorded, array 1150 can be maneuvered within the chamber, such that the positions of electrodes 1151 gather data from a greater volume of the chamber (e.g. “cover” a greater volume of the chamber) as compared to the volume covered if array 1150 was not maneuvered during the recording. In some embodiments, array 1150 is maneuvered such that at least 25%, at least 40%, and/or at least 60% of the volume of the chamber is covered during the recording, such as at least 70% or at least 85%. In some embodiments, array 1150 is maneuvered slowly (e.g. steadily, limiting rapid motion of array 1150). In some embodiments, array 1150 is maneuvered in a pattern. The pattern can be a defined pattern, such as a robotically controlled pattern and/or a pattern known by (e.g. taught to) an operator of system 10 (e.g. one or more clinicians or other operators that performs, or assists in the performance of, diagnostic and/or therapeutic procedures on a patient using system 10). Alternatively or additionally, the pattern can be an operator-determined pattern (e.g. maneuvers performed during recording and/or other patterns determined at the time of the procedure), such as a pattern determined with visual feedback provided by system 10 to the operator. The visual feedback can provide the operator information in a closed loop fashion, such as to assist the operator in completing one or more tasks and/or desired goals of the procedure. For example, the visual feedback can provoke the operator to perform an action such as: expand, extend, and/or alter the operator-determined pattern to include regions with lack of data, insufficient data, and/or poorly spatially distributed data; to increase data quantity and/or quality in a specific region of interest; to replace data in a region (e.g. to re-record data in the region); to achieve full “coverage” including high quality data across the full cardiac chamber surface and/or throughout the whole volume being assessed; and combinations of one or more of these. The visual feedback can indicate the quantity and/or quality of data in time and/or across space. For example, visual feedback can indicate one or more of the following conditions: sufficient data in a region (a point, location, area, and/or volume); insufficient data in a region; coverage (data present or not present) over a region; data density in a region; spatial distribution of data without or with ‘artifacts’ (such as measurement anomalies and/or errors); data consistency or stability over time (within a specific region or regardless of location); and combinations of one or more of these.

The visual feedback provided by system 10 can comprise one or more visual elements, such as points, lines, arrows, mesh, charts, meters, plots, and the like. The visual elements can possess one or more variable characteristics, such as characteristics selected from the group consisting of: size, thickness, color, hue, texture, gradients, translucency, brightness, and combinations thereof, where variation of the characteristic is used to provide additional feedback.

The maneuvering pattern can comprise a repeating and/or a non-repeating set of sub-patterns (“patterns” herein). These patterns in which array 1150 is maneuvered can be configured such that one or more portions of the chamber are covered multiple times and/or for a percentage of the recording time, such as at least 15%, or such as at least 20% of the recording time. In some embodiments, data is recorded continuously over a time period of at least 60 seconds, such as at least 90 seconds, or at least 120 seconds.

In Step 120, the recorded data is analyzed, such as by one or more algorithms 5055 and/or processors 5050 of console 5000. Over the time period of the recorded data, the cardiac cycles are identified and correlated to the recorded data. In some embodiments, system 10 analyzes at least one data signal recorded from a reference catheter, such as a coronary sinus catheter, 1200 and/or from one or more ECG electrodes 560, such as to identify (e.g. and correlate) cardiac cycles. In some embodiments, system 10 analyzes data recorded from two, three, or more “channels” (e.g. data recorded from two, three, or more individual electrodes). In some embodiments, an algorithm of system 10 analyzes the multiple channels of data and determines an optimal channel (e.g. an optimal electrode) for cardiac cycle identification using a stable timing reference. In some embodiments, the optimal channel can be selected based on the electrical signals recorded by that channel, such as by the consistency of detected timing of features of the electrical signal of that channel, and/or by the amplitude of the electrical signal of that channel (e.g. the more easily measured amplitudes). In some embodiments, the optimal channel is selected based on the operator's visual inspection of a graphical display of one or more channels of recorded data. In some embodiments, system 10 presents a graphical display to the operator with a subset of the total number of recorded channels such that that the operator can visually inspect to select the optimal channel. The subset can be selected based on an algorithmic analysis of the recorded data performed by system 10, such as to eliminate one or more sub-optimal channels. In some embodiments, system 10 can filter one or more of the recorded electrical signals, such as with a V-wave filter (also known as QRS filter). For example, system 10 can comprise a filter based on one or more templates for V-wave filtering and/or individual QRS signal filtering (e.g. templates with varying window sizes for one or more QRS signals). In some embodiments, a V-wave filter comprising V-wave blanking (also known as V-wave subtraction, V-wave removal, or V-wave exclusion) can be used, such as V-wave blanking as described in applicants co-pending application U.S. patent application Ser. No. 16/097,959, titled “CARDIAC MAPPING SYSTEM WITH EFFICIENCY ALGORITHM”, filed Oct. 31, 2018, the content of which is incorporated herein in its entirety for all purposes. Alternatively or additionally, system 10 can selectively process one or more of the recorded electrical signals, such as when system 10 identifies a known or expected electrical event and processes intervals of the signal corresponding to the events using a specific and different set of steps (such as removing, subtracting, reducing, retaining, amplifying, or filtering the specific events). In some embodiments, system 10 identifies the T-wave in the signals (corresponding to ventricular repolarization) and eliminates the T-wave morphology or excludes the T-wave interval when processing cardiac information from the atria. In some embodiments, system 10 identifies the P-waves in the signal (corresponding to electrical activity in the atria) and excludes intervals of the signals containing P-waves when processing cardiac information for the ventricles.

In some embodiments, system 10 comprises a “detect-reject” algorithm (e.g. algorithm 5055 described hereabove in reference to FIG. 1). The detect-reject algorithm can be configured to identify a signal characteristic that is known to be undesirable (e.g. a characteristic that degrades the quality of the signal and/or accuracy of later method steps). In some embodiments, the detect-rejection algorithm is further configured to modify and/or reject the detection of cardiac cycles during a time duration proximate a signal characteristic identified as undesirable (e.g. a time duration of one second or less, such as a time duration of 20 ms or 100 ms). In some embodiments, these cardiac cycles are excluded from further calculation. In some embodiments, the cardiac cycles that are overlapped and/or interfered with by an undesirable signal characteristic can be corrected and/or modified, such as to be used in subsequent method steps. Examples of undesirable signal characteristics include but are not limited to: aberrant cardiac rhythms and/or rhythm components (e.g. ventricular depolarization or repolarization), respiration artifact, cardiac tissue contact artifact, and/or any electrical activity from the tissue contact.

In Step 130, system 10 analyzes the lengths of the identified cardiac cycles identified in Step 120. If the cycle lengths are determined to be sufficiently consistent, also referred to as “regular” herein (e.g. within a threshold of a length deviation between the identified cycles), method 100 continues to Step 140. If the cycle lengths are determined to be inconsistent, also referred to as “irregular” herein, for example there is too great a variation in length from one cycle to the next system 10 determines the heart rhythm to be irregular, and method 100 is exited in Step 135. In some embodiments, variation from one cycle to the next is not determined to be irregular if cycle length patterns are determined to repeat on a larger scale, for example a pattern of varying cycle lengths, such as first cycle length followed by a second, different cycle length, in a pattern. In some embodiments, all cardiac cycles identified in Step 120, regardless of cycle length regularity, are utilized (that is, Step 130 is bypassed). In some embodiments, different cycle lengths are processed as subgroups if those cycle lengths cluster into subgroups of regularity around distinct values. In other words, multiple cycle lengths can be concurrently present in the recorded data (e.g. interspersed or interleaved among each other) and can be identified to be distinct groups based on separation of cycle length values. In some embodiments, the number of groups of cycle lengths can be algorithmically determined by a clustering technique. In some embodiments, cycle length groups can be added, removed, and/or merged by the operator. In some embodiments, lower-bound and upper-bound thresholds for each group of cycle lengths can be established and adjusted (e.g. algorithmically or manually by the operator) for any group.

In Step 140, the recorded data is segmented by the cardiac cycles determined in Step 120. In some embodiments, the segmented recorded data includes data recorded from one or more of: coronary sinus catheter 1200 (e.g. a coronary sinus catheter or other reference catheter); basket catheter 1100; and/or surface ECG electrodes 560; as well as any two of these, or all three of these. In some embodiments, the recorded data is segmented into segments with a duration of at least a time period equal to the maximum difference between activation times recorded by two or more (e.g. all) electrodes of a reference catheter (e.g. the data is segmented using “narrow band clustering”).

In optional Steps 150 and 160, the recorded data can be filtered to remove unwanted data, such that the unwanted data is not incorporated in the electrical activity model of method 100. In Step 150, segmented data can be filtered by the cycle length, for example such that segments with a cycle length variation exceeding a threshold are removed or are processed as a separate subgroup. In some embodiments the threshold is determined based on an average of the cycle lengths determined in Step 120, as described hereabove. In Step 160, the recorded data can be analyzed, such as by an algorithm and/or processor of system 10 (e.g. algorithm 5055 and/or processor 5050 of console 5000, respectively), to filter out inconsistent, erroneous, and/or otherwise unwanted data. In some embodiments, unwanted data is caused by recording from a malfunctioning electrode (e.g. a malfunctioning electrode 1151, 1251, and the like). If a malfunctioning electrode is identified, data recorded from that electrode can be selectively removed from the recorded data altogether, or for example, only after the instance a malfunction occurs (e.g. if an electrode “stops working” during the recording). In some embodiments, Step 160 can comprise a filtering and/or outlier-detection process that processes the data (e.g. combines and/or removes data) based on the similarity of signals.

In Step 170, the segments of the recorded data are time-aligned, such as to generate a composite recording (a composite of one or more time-aligned segments) over the duration of a single cardiac cycle. In some embodiments, “pattern clustering” is performed, and the segments are grouped based on one or more identified patterns (e.g. patterns of electrical activity) and/or characteristics. In some embodiments, segments are differentiated and/or grouped based on signal characteristics such as cardiac cycle length, signal morphology (e.g. comparison based on cross-correlation and/or wavelet decomposition), amplitude, frequency, frequency components, and/or wavelet analysis. In some embodiments, a clustering algorithm is used, such as an algorithm comprising a cluster model-based algorithm. For example, a cluster model-based algorithm can comprise one or more of the following: a connectivity model-based algorithm (such as a hierarchical clustering algorithm) wherein models are based on distance connectivity; a centroid model-based algorithm (such as a k-means clustering algorithm) that can represent each cluster by a single mean vector; a split and merge based algorithm wherein the data is initially split into many clusters and then merged based on affinity; a density model-based algorithm that defines clusters as connected dense regions in the data space; a distribution model-based algorithm (such as a Gaussian mixture model clustering algorithm) wherein clusters are modeled using a statistical distribution (such as a multivariate normal distribution); a graph-based model algorithm; a neural model-based algorithm (such as a self-organizing map and/or other unsupervised neural network) wherein an artificial neural network and/or other non-linear statistical data modeling tool can be used to model complex relationships and patterns in data; and combinations of one or more of these. In some embodiments, data projection and/or preprocessing approaches are applied, such as dimension reduction, principal component analysis, and/or multidimensional scaling.

In some embodiments, segments are differentiated (e.g. clustered) based on “template matching,” such as matching to a set of templated (e.g. previously categorized) signal characteristics across one or more channels (e.g. from individual electrodes) from a reference signal. In some embodiments, segments are differentiated based on the consistency of relative time intervals measured by and/or between each of multiple electrodes. For example, a detected fiducial time T1 through T10 can be detected on electrodes E1-E10, respectively. Segments can be grouped by the degree of consistency and/or similarity to a set of times T1′-T10′, a form of a template. Some cardiac rhythms can have more than one set of times T1-T10, recurrently. In some embodiments, fiducial times are detected from differential signals from two or more electrodes (e.g. the differential signal between neighboring electrodes, obtained by subtracting one from the other).

In some embodiments, segments are transformed into a different domain such as wavelet or Fourier, and/or projected to a lower dimension space using linear/nonlinear dimensionality reduction such as T-distributed Stochastic Neighbor Embedding, or Locally Linear Embedding. The segments or transformed segments are clustered using any of the clustering methods mentioned above and/or combinations of them. The clustering is performed without specifying the number of groups in advance or with a known number of groups. Matching a segment to a group is achieved by meeting a required criteria, such as correlation coefficient is higher than a threshold, distance metric such as vector norm, cosine, KL-divergence is lower than a threshold or requiring a statistical metric (such as the mean, median, standard deviation) to satisfy the condition (lower than threshold for distance metric, higher than threshold otherwise).

In some embodiments, segment differentiation is further refined by grouping segments based on a degree of correlation, also referred to as “matching” herein, to the unique signal morphologies measured by each of multiple electrodes (e.g. 10) of the reference catheter. For example, signal morphologies S1 through S10 are identified from signals E1 through E10, respectively, to be a template. A segment with signal morphologies S1′ through S10′ from signals E1-E10 at a different time can be compared to the template, such as by determining the degree of correlation between S1 and S1′, S2 and S2′, and so on, using a mathematical and/or statistical comparison, such as cross-correlation. The set of correlation values can be used to determine if the segment matches the template. Template matching can comprise a method selected from the group consisting of: requiring a set of criteria to be met, such as requiring the correlation value on all signals to exceed a threshold (e.g. X-corr greater than 0.8, or X-corr greater than 0.9); requiring the correlation value on a subset of signals (e.g. more than 60% of signals, or more than 80% of signals) to exceed a threshold; requiring a statistical metric (such as the mean, median, standard deviation) to exceed threshold; and combinations of one or more of these.

In some embodiments, a set of signal characteristics from a single event (e.g. a single cardiac event such as a single cardiac beat) are used as the template to which all other segments are matched against. In some embodiments, the template is updated at a fixed and/or variable time interval. In some embodiments, as one segment is found to be unmatched to the existing template, that segment is used as a second template to form a second group. In these embodiments, as additional segments are found to be unmatched to any of the existing templates, they can be used to expand the set of templates to form additional groups. In this way, the set of templates can continue to expand throughout the segment collection process.

In some embodiments, one or more templates and their respective groups are merged and/or combined to form a combined group of all matching segments. Combining can be done manually by the operator and/or automatically by an algorithm based on the similarity between the templates and/or common difference from all remaining templates. In some embodiments, the system visually displays the unique groups (e.g. any set of signals or recorded data from the groups, from any subset of catheters) to assist the operator in merging groups and/or deciding on a preferred group to continue processing. In some embodiments, the visual display of a group of segments includes a user interface that allows the operator to use a user input device to manually exclude subsets of signals in the group and/or select the subset of signals in the group to form a new, unique group.

In some embodiments, an algorithm of system 10 (e.g. algorithm 5055) is configured to assess the degree of matching of a segment to a template based on a set of criteria. For example, the algorithm can compare the degree of matching to a threshold. In some embodiments, the criteria is dynamically adjusted to be more restrictive or less restrictive to successfully match a segment to a template. In some embodiments, as the criteria are adjusted, previously matched segments are reprocessed to regenerate unique templates and groups. In some embodiments, as the criteria are adjusted (e.g. if the criteria has become more restrictive), only the templates representing each group can be processed using the adjusted criteria to determine if groups should be combined. In some embodiments, as the criteria are adjusted (e.g. particular if the criteria become less restrictive), only the segments within an individual group need to be reprocessed against new, unique templates arising from the group based on the adjusted criteria to determine if groups should be sub-divided. In some embodiments, two or more of the methods, described herein, of clustering, templating, matching, and/or any other method of differentiating beats or rhythms are used in combination (e.g. sequentially or simultaneously).

After pattern clustering, two, three, or more composite recordings can be generated, each comprising a unique pattern of activation (e.g. a pattern of activation over a cardiac cycle). Alternatively or additionally, after pattern clustering, the cluster with the greatest percentage of segments can be chosen as the (single) composite recording. In some embodiments, any cluster with a percentage of segments exceeding a threshold can be identified for generation of a composite recording (e.g. one, two, or more clusters), such as when clusters below the threshold are discarded. In some embodiments, a composite recording can be generated based on data comprising one, two, or more cycle lengths. In some embodiments, for example for narrow band clustering, the input segment width for a composite map to be generated is not defined. In these embodiments, data comprising two cycle lengths can be used to determine activation activity around the segment boundary (e.g. since there are multiple inversions within data over two cycle lengths).

In Step 180, the cardiac electrical activity during the simulated cardiac cycle is modeled based on the composite recording. In some embodiments, the cardiac electrical activity is modeled by indexing the cardiac electrical data, such as by indexing a representative time and/or amplitude for one or more locations on the cardiac anatomy. In some embodiments, the cardiac electrical data is modeled by interpolating and/or projecting cardiac electrical data from a set of locations to a separate set of locations. In some embodiments, the cardiac electrical activity is calculated via measurements made using electrodes positioned in contact with tissue. In these embodiments, the cardiac electrical activity can comprise voltage-based measurements. In some embodiments, cardiac electrical activity is calculated using forward modeling and/or inverse modeling (e.g. via non-contact recordings which can be made with or without contact recordings). Alternatively or additionally, the cardiac electrical activity can be calculated using an inverse solution. In some embodiments, the cardiac electrical activity is analyzed using methods and techniques described in applicants co-pending applications: U.S. patent application Ser. No. 16/014,370, titled “METHOD AND DEVICE FOR DETERMINING AND PRESENTING SURFACE CHARGE AND DIPOLE DENSITIES ON CARDIAC WALLS”, filed Jun. 21, 2018; U.S. patent application Ser. No. 16/242,810, titled “EXPANDABLE CATHETER ASSEMBLY WITH FLEXIBLE PRINTED CIRCUIT BOARD (PCB) ELECTRICAL PATHWAYS”, filed Jan. 8, 2019; the content of each of which is incorporated herein by reference in its entirety for all purposes, such as to produce dipole density and/or surface charge density data. In some embodiments, the cardiac electrical activity can be calculated using an automatic timing annotation algorithm (e.g. an algorithm that identifies signal characteristics, such as those corresponding to cardiac tissue depolarization, activation, and/or repolarization). In some embodiments, the analysis of the cardiac electrical activity can be displayed to an operator, such as with an activation timing map produced on a display. In some embodiments, the analysis of the cardiac electrical activity can be displayed as described in applicants co-pending applications U.S. patent application Ser. No. 16/097,955, titled “CARDIAC INFORMATION DYNAMIC DISPLAY SYSTEM AND METHOD”, filed Oct. 31, 2018, the content of which is incorporated by reference in its entirety for all purposes, such as when the activity is displayed as dipole density maps, surface charge maps, voltage maps, activation timing maps, isochronal maps, and/or Laplacian amplitude maps.

In some embodiments, Steps 120-180 of Method 100 are performed during a clinical procedure, for example, where calculating the model of cardiac electrical activity is performed in “real time” (e.g. such that the model is provided to the operator in near real time, such as within a few milliseconds, seconds, or minutes, of the data being recorded). Alternatively or additionally, Steps 120-180 can be performed at a time after the completion of the clinical procedure during which the biopotential data recorded in Step 110 was recorded.

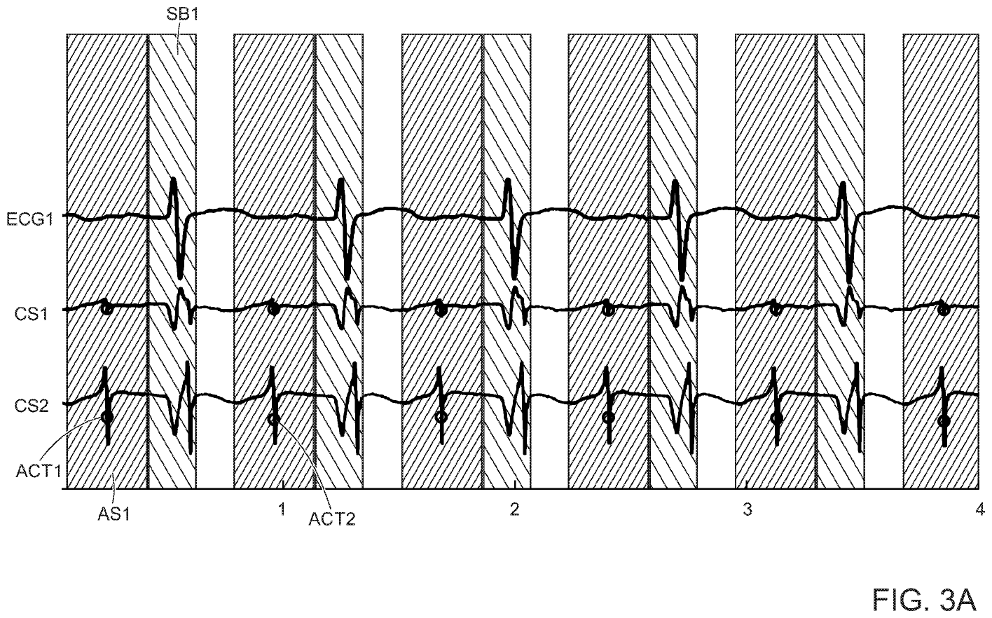

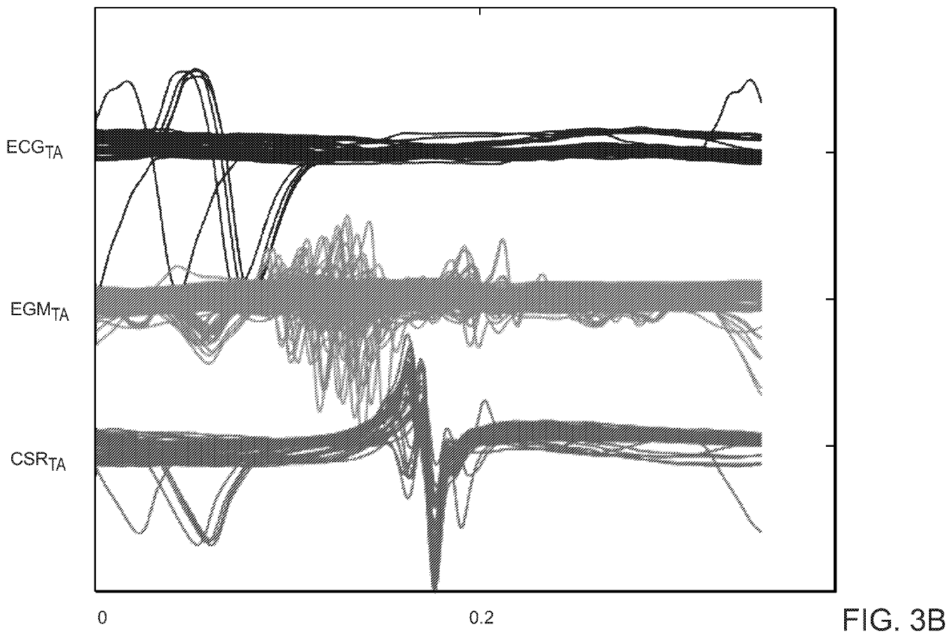

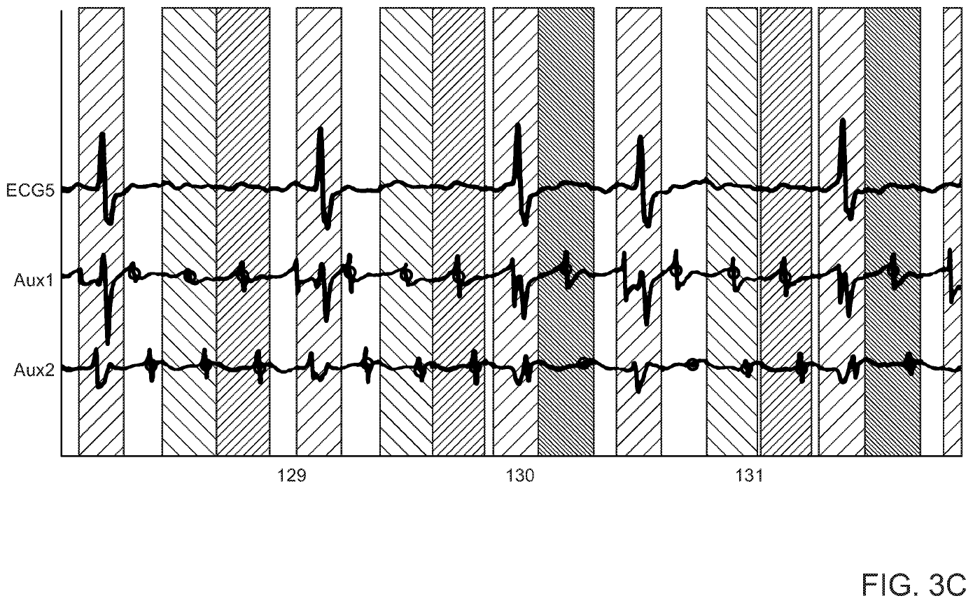

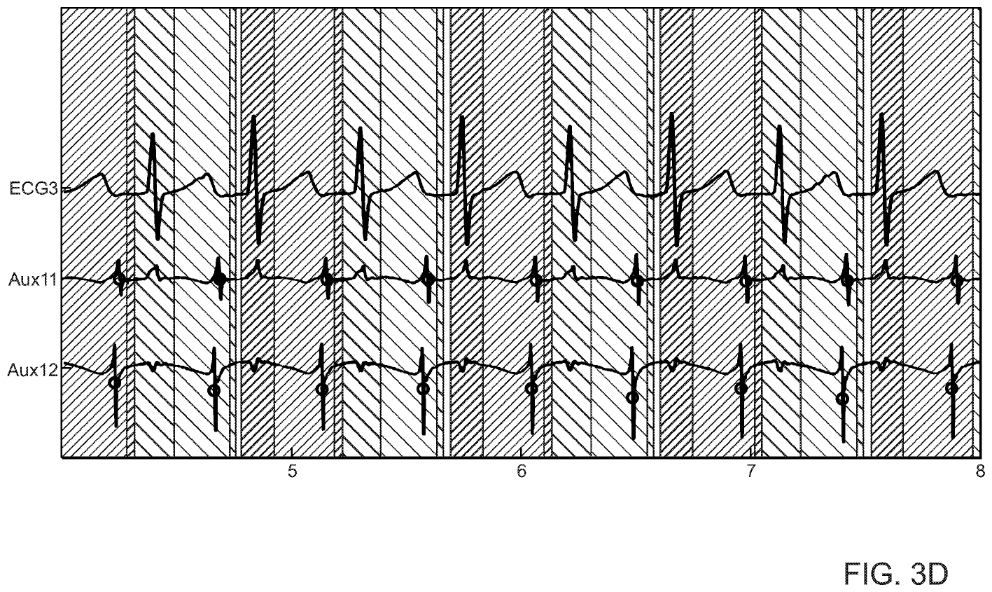

Referring now to FIG. 3A, a graph of recorded electrical activity is shown, consistent with the present inventive concepts. FIG. 3A depicts signals recorded on three channels of system 10, a first signal ECG1, recorded from an ECG lead attached to console 5000, and a second and third signal CS1 and CS2, respectively, recorded from two electrodes 1251 of coronary sinus catheter 1200. FIG. 3A illustrates exemplary signals analyzed by system 10, such as in steps 120 and/or 140 of method 100 described hereabove in reference to FIG. 2. In some embodiments, system 10 is configured to record two, three, or more ECG signals, recorded from internal and/or external patient locations (e.g. one or more internal electrodes and/or one or more external electrodes). Additionally, system 10 can be configured to record multiple signals from coronary sinus catheter 1200 (e.g. from multiple electrodes of catheter 1200), such as 10 or 12 signals from 10 or 12 electrodes 1251. FIG. 3A depicts an illustrative subset of these recorded signals.

In some embodiments, system 10 is configured to analyze signal ECG1 to identify the V-wave, the T-wave, and/or the P-wave, such as for V-wave, T-wave, and/or P-wave blanking (also known as subtraction or removal), as described hereabove in reference to FIG. 2. For illustrative purposes, V-wave filtering is shown by shaded segment SB1.

In some embodiments, system 10 is configured to compare signals recorded from each electrode 1251 of coronary sinus catheter 1200, to identify the channel with optimal recorded data (e.g. data with the most clearly identifiable characteristics), and to select that channel as the reference channel. For example, as shown in FIG. 3A, signal CS1 comprises a relatively low amplitude recording representation of the characteristics of the cardiac cycle, whereas CS2 comprises a relatively high amplitude recording of those characteristics. As shown, activations ACT1 and ACT2 are more clearly identifiable on signal CS2, and as such, signal CS2 would be selected as the reference signal. In some embodiments, system 10 is configured to compare signals recorded from each electrode of a multi-electrode catheter and/or multiple catheters positioned in one or more locations of the heart, for example in the right and/or left atria, the right and/or left ventricles, in the pulmonary outflow tract, and/or any of the circulatory (e.g. venous) structures of the heart. In some embodiments, system 10 is configured to compare signals recorded from one or more electrodes placed in one or more locations on the body surface, including for example any or all of the ECG electrodes, impedance-driving patch-electrodes, and/or any other auxiliary electrode placed on the body for measuring signals generated by the heart. In some embodiments, an algorithm of system 10 (e.g. an algorithm 5055 as executed by processor 5050 of console 5000) analyzes the recorded signals and determines the proper reference signal. Alternatively or additionally, system 10 can display one or more signals, such as signals CS1 and CS2, and an operator of system 10 can assess and manually select the proper reference signal. Shaded region AS1 depicts the identified active segment, based on activation ACT1.

Referring additionally to FIG. 3B, a graph of time-aligned recorded electrical activity is shown, consistent with the present inventive concepts. As described hereabove in reference to step 170 of method 100, the recorded signals can be time-aligned, such as by aligning activations identified on a selected reference signal (e.g. signal CS2 described hereabove in reference to FIG. 3A). FIG. 3B provides a graphical representation of multiple time-aligned ECG signals, signals ECG_{TA}, multiple time-aligned coronary sinus reference signals, signals CSRTA, and multiple time-aligned signals recorded from multiple mapping electrodes 1151, signals EGM_{TA}. As described herein, signals EGM_{TA} can be analyzed by system 10 (e.g. by algorithm 5055 and/or processor 5050 of console 5000) to produce one or more cardiac electrical activity maps.

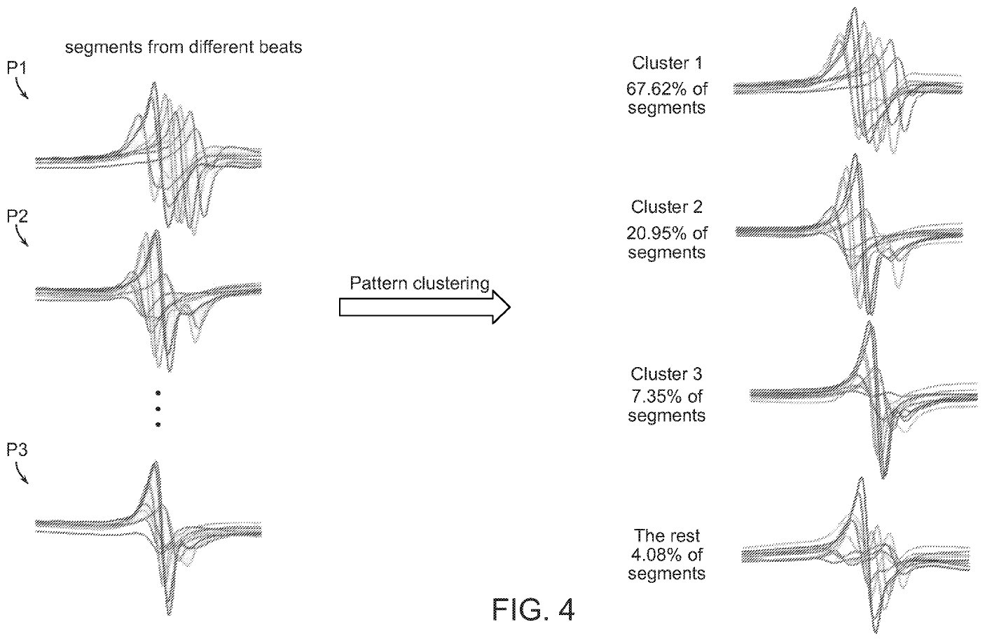

Referring now to FIG. 4, a visual representation of pattern clustering is shown, consistent with the present inventive concepts. As described hereabove in reference to step 170 of method 100, system 10 can be configured to group two or more segments of recorded data with a pattern clustering algorithm (e.g. an algorithm 5055 executed by processor 5050 of console 5000). System 10 can analyze the segments identified in the data and/or identify one or more patterns in that data. For example, three patterns, P1, P2, and P3, are illustrated in FIG. 4. Patterns can comprise activation waveforms as recorded from electrodes 1151 for a period of time (e.g. over the period of a single activation or cardiac cycle). As an illustrative example, as shown by pattern P1, the waveforms as recorded by each electrode 1151 comprise more temporal variation, while the waveforms as recorded by each electrode 1151, shown by pattern P2, comprise less temporal variation.

After the identification of two or more patterns, system 10 can be configured to group (also referred to as “cluster” herein) the segments based on these identified patterns. Again, as an illustrative example, system 10 can cluster the recorded data (e.g. segments) into four clusters, Cluster 1 representing the data that matched pattern P1, Cluster 2 representing the data that matched pattern P2, Cluster 3 representing the data that matched pattern P3, and a fourth grouping, representing the segments that did not match any of patterns P1, P2, or P3. In some embodiments, a pattern can comprise a unique wave morphology and/or activation pattern. In some embodiments, this fourth grouping represents a percentage of segments below a threshold, such that an additional pattern is not identified by system 10. In some embodiments, only a pattern identified and determined to represent a population of segments over a percentage threshold will be identified for clustering. In some embodiments, the percentage threshold is greater than 5%, such as greater than 10% or 15%. In some embodiments, system 10 is configured to only identify one, two, or three patterns, such as the one, two, or three dominant patterns, regardless of the overall percentage of the population of segments. In some embodiments, the pattern clusters can be visually identified in a time-course of signals as recorded, such as shown in FIG. 3C wherein each unique color identifies segments within the same cluster. In some embodiments, a user interface can be configured to select a cluster of segments by selecting a correspondingly colored area and cardiac information corresponding to the selected cluster can be displayed. In some embodiments, the clusters may each have a unique cycle length, as shown in FIG. 3D (shown is a bigeminal rhythm wherein the cycle length varies between two values).

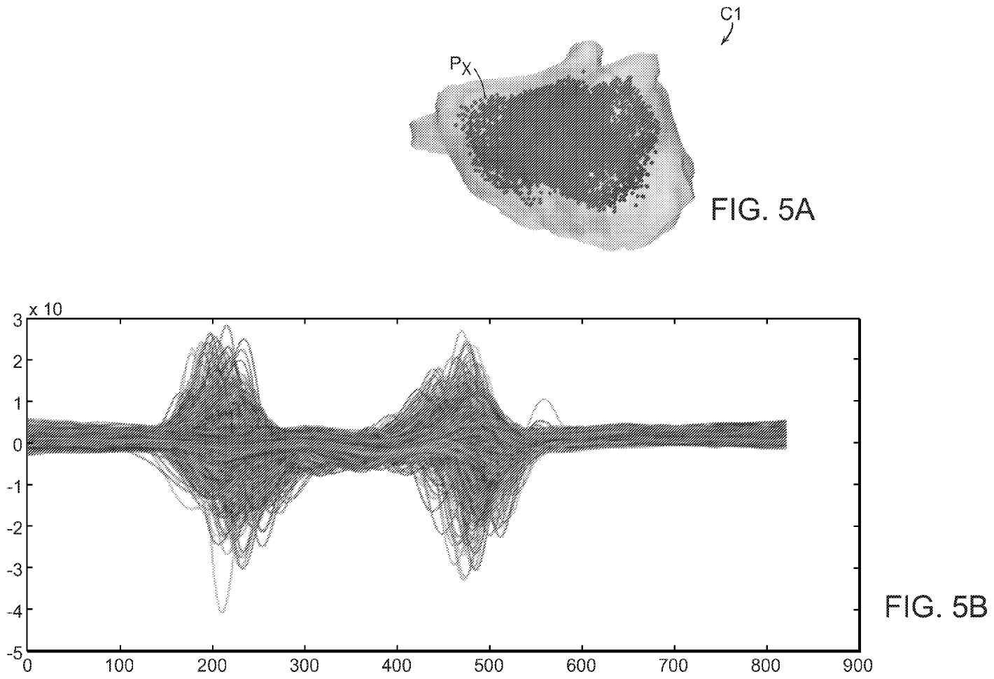

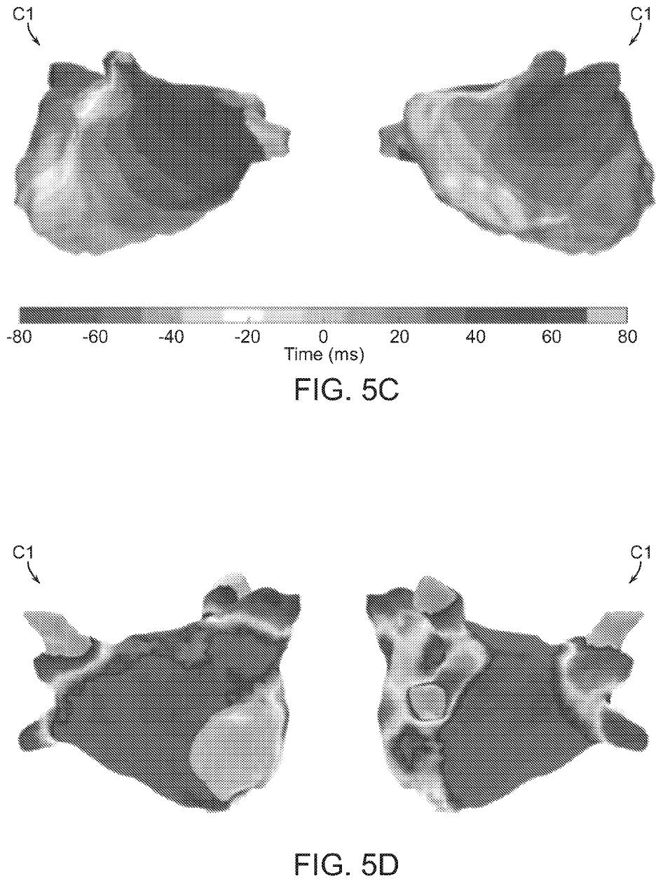

Referring now to FIGS. 5A, 5B, 5C, and 5D, a graphical representation of a cardiac chamber with multiple recording locations, a graph of multiple cardiac electrical activity recordings, an activation map, and an amplitude map are illustrated, respectively, consistent with the present inventive concepts. FIG. 5A depicts a model of a cardiac chamber C1, and points Px within the chamber C1. Each point Px represents the physical location of an electrode 1151 within chamber C1 while a segment of data was recorded by that electrode (e.g. while a segment of a signal was recorded, such as a signal recorded over a period of time greater than the length of the segment). For example, if mapping catheter 1100 comprises 48 electrodes 1151, and 100 segments are included in the illustrated embodiment (e.g. 100 segments of biopotential signals, segmented by cardiac cycles, the 100 segments included in a composite recording), 4800 points Px would be illustrated (assuming no filtering, for example for malfunctioning electrodes). In some embodiments, mapping catheter 1100 comprises at least 48 electrodes 1151, or at least 16 electrodes, or at least 10 electrodes, or at least 4 electrodes. As described hereabove in reference to step 110 of method 100, electrode array 1150 has been maneuvered within chamber C during this recording, such that the points Px are distributed throughout the chamber (e.g. the electrodes 1151 cover a large volume of chamber C1 and/or surface area of chamber C1). In some embodiments, points Px within chamber C1 are points recorded when the recording electrodes are in contact and/or are proximate (e.g. less than 5 mm) the chamber wall. In some embodiments, points Px within chamber C1 are points recorded when the recording electrodes are not in contact with the chamber wall, such as electrodes that are positioned at a location greater than a threshold distance (e.g. >5 mm) from the chamber wall. In some embodiments, all points Px within chamber C1 are used irrespective of the state of contact of the recording electrodes. In some embodiments, points in contact and points not in contact are processed separately, and the outputs of each generated model of cardiac activation are merged, integrated, blended, and/or otherwise combined.

In FIG. 5B, a graph of the electrical activity recorded by electrodes 1151, (e.g. a composite recording as described hereabove in reference to step 170 of method 100) is illustrated. In the example above, 4800 signals would be represented in FIG. 5B. Each electrode 1151 can record biopotential data comprising biopotential signals and the biopotential signals can be segmented by cardiac cycles into multiple biopotential signal segments. As described herein, one or more composite recordings can comprise two or more of the multiple biopotential signal segments. In some embodiments, the total number of recorded biopotential signal segments can comprise more than 500 biopotential signal segments, such as more than 1,000, more than 2,000, more than 5,000, or more than 10,000. In FIG. 5C, an activation map is illustrated, such as a local activation time (LAT) map, representing the pattern of activation calculated from the data shown in FIG. 5B. In FIG. 5D, an amplitude map is illustrated, such as a voltage amplitude map or a normalized charge density amplitude map, representing the amplitude of cardiac electrical activity at various locations on the anatomy calculated from the data shown in FIG. 5B. As described hereabove in reference to step 170 of method 100, an activation map can be generated from the composite recording illustrated in FIG. 5B, such as using an automatic annotation and/or inverse solution, as described hereabove.

System 10 can display the composite maps using a visual display. The visual display can be displayed continuously and it can be updated dynamically. In some embodiments, the “map” (e.g. cardiac electrical information) can be dynamically updated. Additionally or alternatively, the data quality and/or quantity information can be dynamically updated as the operator collects new data (e.g. all processing and display steps repeat on a loop while the operator actively collects). Alternatively or additionally, the display can show only the data quality and/or quantity information while the operator collects the data, and the map can be processed and displayed after the data collection is complete. In some embodiments, the system is configured to process all or a portion of the steps described hereabove in the background, and it can provide feedback to the operator while data is being recorded (e.g. operating in a closed loop configuration). For example, system 10 can automatically inform the operator of one or more of the following: when some of the criteria for creating a composite map are currently being achieved (e.g. reference catheter criteria for stability, pattern clustering, template matching, and/or any of the aforementioned criteria are being achieved); suggestions to the operator that the operator can complete a composite map by completing a particular set of suggested steps (e.g. moving to additional locations for continued recording); and combinations of one or more of these. In some embodiments, system 10 displays the map to the operator, at least partially, during data collection. Alternatively or additionally, system 10 can display the map to the operator following completion of the data collection. In some embodiments, previously recorded data can be post-processed using one or more methods of the present inventive concepts to produce a map. In some embodiments, the previously recorded data can be collected when performing a separate task (such as tracing and/or scanning the anatomy with ultrasound).

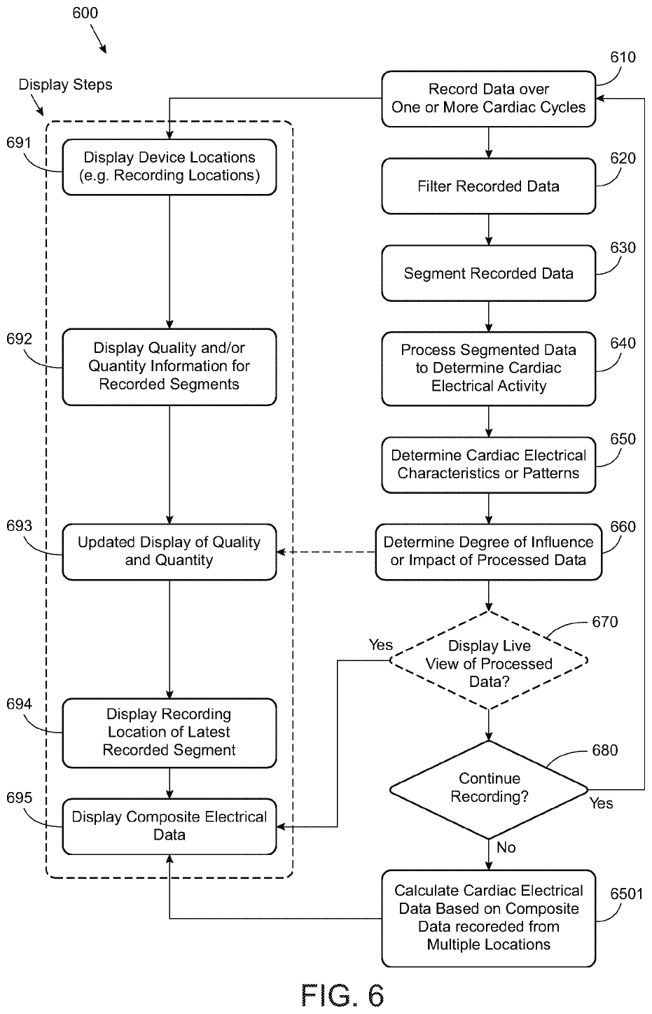

Referring now to FIG. 6, a flowchart of a method for recording and modeling electrical activity of a patient is illustrated, consistent with the present inventive concepts. Method 600 of FIG. 6 is described using various components of system 10 of FIG. 1 as described hereabove.

In Step 610, data is recorded (e.g. by console 5000) by recording elements (e.g. electrodes and/or ultrasound transducers) of one or more catheters 1000 inserted into a patient P. The data can be recorded over one or more cardiac cycles. In some embodiments, data is recorded from a catheter including an array of recording elements positioned on a basket and/or other structure, such as mapping catheter 1100 described herein. Data can also be recorded from auxiliary catheters, such as a reference catheter, such as coronary sinus catheter 1200. For example, the distal portion of catheter 1200 (e.g. at least electrode array 1250) can be positioned within the coronary sinus of the heart H of patient P. Electrode array 1150 (e.g. a basket array comprising electrodes 1151) of catheter 1100 can be positioned within a chamber of heart H, such as within the left atrium of heart H. Data (e.g. biopotential data and/or localization data) can be recorded from both catheter 1100 and 1200. As the data is recorded, array 1150 can be maneuvered within the chamber, such that the positions of electrodes 1151 gather data from a greater volume of the chamber (e.g. “cover” a greater volume of the chamber) as compared to the volume covered if array 1150 was not maneuvered during the recording. In some embodiments, array 1150 is maneuvered slowly (e.g. steadily, limiting rapid motion). In some embodiments, array 1150 is maneuvered in a pattern. The pattern can be a defined pattern, such as a robotically controlled pattern and/or a pattern known by (e.g. taught to) and performed by the operator. Alternatively or additionally, the pattern can be an operator-determined pattern (e.g. determined at the time of the procedure), such as a pattern generated by the operator using visual feedback provided by system 10 (e.g. via a video monitor or other display). The provided visual feedback can assist the operator in completing one or more tasks or goals, for example: to expand, extend, and/or alter the operator-determined pattern to regions with lack of data, insufficient data, and/or poorly spatially distributed data; to increase data quantity and/or quality in a specific region of interest; and/or to replace data in a region. The visual feedback can be used to indicate the quantity and/or quality of data in time and/or across space. For example, visual feedback can be used to indicate one or more of the following: sufficient data in a region (e.g. a point, location, area, or volume); insufficient data in a region; coverage (e.g. data present or not present) over a region; data density in a region; spatial distribution of data without or with ‘artifacts’ (such as measurement anomalies or errors); and data consistency and/or stability over time (e.g. within a specific region or regardless of location).

The visual feedback can comprise one or more visual elements that can be presented to the operator (e.g. on a display of system 10, such as to provide feedback), such as points, lines, arrows, mesh, charts, meters, plots, and the like. The visual elements can possess one or more attributes that are varied (e.g. varied to provide feedback), such as an attribute selected from the group consisting of: size; thickness; color; hue; texture; visual gradient; translucency; brightness; and combinations of these. The pattern can comprise a repeating or a non-repeating set of sub-patterns (“patterns” herein). These patterns in which array 1150 is maneuvered (e.g. robotically and/or by the operator) can be configured such that one or more portions of the chamber are covered multiple times and/or for a percentage of the recording time, such as at least 15%, or such as at least 20% of the recording time. In some embodiments, data is recorded continuously over a time period of at least 10 seconds, such as at least 30 seconds, or such as at least 90 seconds. In some embodiments, visual feedback is provided to the operator in Steps 691-693, described herebelow. In some embodiments, one or more additional recordings can be performed to collect additional data to be processed (e.g. in combination with the prior recordings). In some embodiments, the recording electrodes can be changed to an alternate set of electrodes (e.g. alternate electrodes on the same and/or another device). In some embodiments, devices with different numbers and configurations of electrodes can be used to record the data for processing.

In Step 620, the recorded data is filtered to remove or at least reduce noise, artifacts, and/or other erroneous data. Data filtering can correct for data errors including electrical interference, artifacts from mechanical motion, artifacts caused by electrode contact with tissue, temporary or permanent disconnection of a measurement electrode, and combinations of one or more of these. In some embodiments, system 10 can filter one or more of the recorded electrical signals as described hereabove in reference to Step 120 of FIG. 2.

In Step 630, a portion of the recorded data is segmented for processing. In some embodiments, the segmented portion can be as short as a single time sample or as long as several hours, such as between 100 msec and 3sec, such as 100 msec, 200 msec or 400 msec. In some embodiments, the length of the data segment is governed by the stability of the measurement device. For example, the length of the segmented portion of data can be automatically determined and selected for processing based on periods of minimal motion of the recording electrode array. In some embodiments, the system determines that the recording electrode array has remained stable in a region (e.g. a specific volume within a cardiac chamber) for an amount of time by determining the time when the array was moved into the region and when the array was moved out of the region.

In Step 640, the recorded data is analyzed, such as by one or more algorithms 5055 and/or processors 5050 of console 5000. The recorded data can be processed using an inverse solution to solve for cardiac electrical activity information. In some embodiments, electrical activity information comprises raw surface charge density data (e.g. dipole density data), surface voltage data, and/or activation time data.

In Step 650, cardiac electrical characteristics are calculated using the cardiac electrical activity information determined in Step 640. In some embodiments, conduction velocity (e.g. magnitude, direction, and/or both), and/or one or more conduction patterns are calculated. The one or more identified conduction patterns can comprise conduction patterns described in applicants co-pending United States Patent Application serial number 16/097,959, titled “CARDIAC MAPPING SYSTEM WITH EFFICIENCY ALGORITHM”, filed Oct. 31, 2018, the content of which is incorporated herein by reference in its entirety for all purposes. In some embodiments, indices of complexity are interpreted from one or more of the following: detected conduction patterns; surface charge density and/or voltage electrograms; detected activation times; calculated conduction velocity and/or relative conduction velocity change; other physiologic measurements related to cardiac rhythm; and combinations of one or more of these. In some embodiments, two or more electrical characteristics (or patterns of characteristics) detected in one or more unique locations of the cardiac tissue dynamically engage each other, also referred to as “couple” herein, such as when engaged simultaneously and/or sequentially in time.

In Step 660, based on the location of the measurement device (e.g. the recording electrode array), the cardiac information measured from any location is determined to have a degree of influence and/or impact on the processed cardiac electrical activity. Of the processed cardiac electrical activity data as calculated from measurements made from an electrode location L1 (a location on or within the cardiac chamber), electrical activity data at one or more locations on the cardiac chamber (locations the same as or different than L1) can be included, excluded, and/or weighted preferentially or non-preferentially. For example, if the catheter is near a central and/or balanced location within the chamber, the cardiac electrical information can be uniformly weighted to a moderate value moderate (e.g. 0.5 on a 0 to 1 scale). If the catheter is in close proximity to a chamber wall, the closer tissue areas (e.g. at a distance less than 40 mm) can be weighted higher (e.g. 0.9 on a 0 to 1 scale) while farther tissue areas (e.g. at a distance greater than 40 mm) can be weighted lower (e.g. 0, or 0.1). Weighting can comprise a gradient as a function of distance. This weighting can be used to emphasize data recorded from closer positions, where the inverse solution can have improved accuracy. Other criteria to determine weighting factors can be solid-angle to a region of tissue (e.g. more en face is weighted higher, while more oblique is weighted lower). Weights can be made binary, 0 or 1, such as to use a strict include vs. exclude model. The weighting algorithm can be automatically applied by system 10. Alternatively or additionally, weights can be manually adjusted. In some embodiments, the influence of the processed data is visually indicated to the operator during and/or after Step 660 in Step 693, described herebelow.

In Step 670, system 10 can be configured to display a “live” view of the calculated electrical activity data (e.g. in real time or at least near real time, as the data is calculated). If system 10 is configured to display live data, Step 695 is executed, as well as step 680. If system 10 is not configured to display live data, process 600 continues only to Step 680.

In Step 680, if the operator continues to record data, process 600 continues to Step 610, and process 600 is repeated while data is recorded. If data is no longer being recorded, process 600 continues to Step 6501.

In Step 6501, cardiac electrical data is calculated using the cardiac electrical activity information determined throughout one or more iterations of process 600. Cardiac electrical data calculated in Step 6501 comprises composite data based on data recorded from one or more locations, over one or more recording time periods. After 6501, process 600 continues to Step 695.

In Step 695, composite cardiac electrical data determined during process 600 is displayed to the operator. In some embodiments, Steps 691-695 (“Display Steps” as shown) are performed simultaneously with one or more Steps 610-690, such as is referenced hereabove.

In Step 691, one or more locations of one or more recording devices are displayed to the operator relative to a model of the cardiac anatomy (e.g. the location of the recording devices during a recording).

In Step 692, the quality and/or quantity of information recorded can be visually displayed to the operator. For example, relative to a model of the cardiac anatomy, one or more indicators can be used to inform the operator of adequate recording in one or more regions. When adequate recordings have been made in all desired regions, a “go/no-go” indicator (such as a colored light) can be displayed to the operator.

In Step 693, the composite, weighted data is displayed to the operator. The display can summarize all data after completion of all, or at least a portion of, data collection. Alternatively or additionally, the display can continuously (e.g. dynamically) display the weighted data as it is recorded and calculated. The display can also include and/or overlay additional visual information to assist the operator in optimally collecting the data and/or maneuvering the recording device. For example, a display can indicate that the data is of poor quantity and/or quality in a region, such that maneuvering the catheter towards that region can improve the quantity or quality of data collected. In some embodiments, the composite, weighted data can be displayed in its original form, that is, using modified, composite, weighted values, but using the same visual properties and behaviors. In some embodiments, the composite, weighted data is processed through a separate algorithm to summarize the data, for instance, to show a summary map of electrical properties or conduction patterns of different regions of the cardiac chamber, or interactions between regions (e.g. coupling), for example by visually emphasizing areas that consistently and stably exhibit specific signal characteristics and/or conduction patterns.

In Step 694, the visual feedback display also provides temporary visual indication that new data has been added to the composite map and from what location the measurements were made. In some embodiments, points at different locations have an altered coloration or other alteration of appearance. In some embodiments, a second visual element provides a visual accent to data at a given location (e.g. a white ring around the location point).

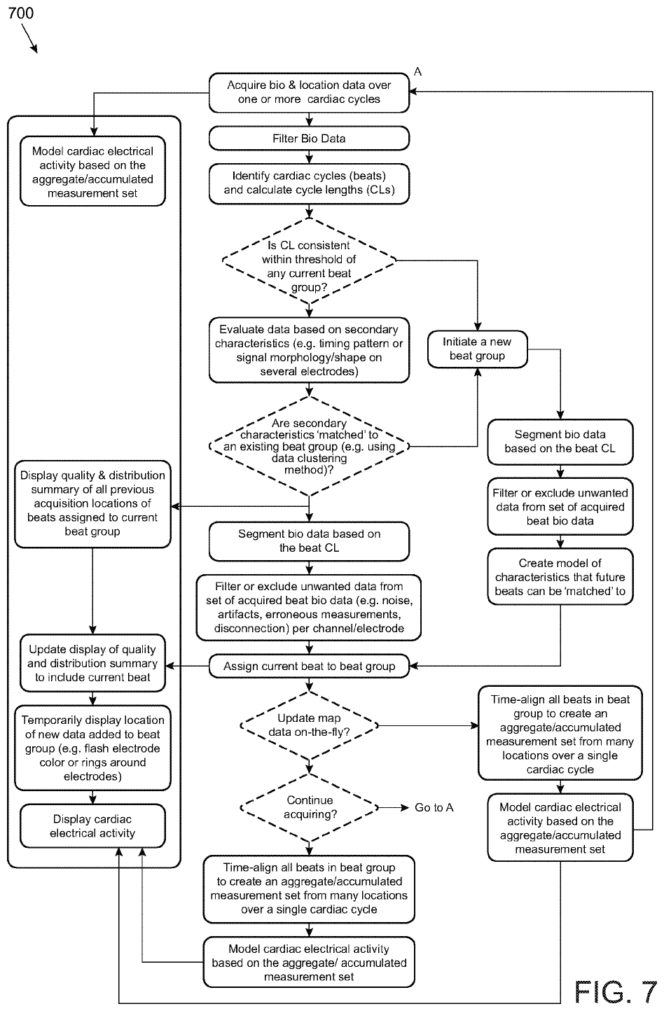

Referring now to FIG. 7, a flowchart of a method for recording and modeling electrical activity of a patient is illustrated, consistent with the present inventive concepts. Method 700 of FIG. 7 can be achieved using various components of system 5000 of FIG. 1 as described hereabove. Method 700 of FIG. 7 can be similar to method 100 of FIG. 2 described hereabove. In some embodiments, method 700 is performed during a clinical procedure, for example, where the cardiac electrical activity is calculated and displayed to the operator of system 10 in real time, or very near real-time. Alternatively or additionally, one or more steps of method 700 can be performed after the completion of the clinical procedure during which the biopotential and location data was recorded.

In some embodiments, the steps of method 700 can be performed in the order illustrated. Additionally or alternatively, the steps of method 700 can be performed in any other order suitable to model and display the cardiac electrical activity, such as is described herein. In some embodiments, one or more beats are grouped based on the morphology of the beats. Subsequently, each group is filtered (e.g. one or more beats are removed from the group) based on cycle length. Finally, if two groups comprise the same cycle length and similar morphologies, the two groups can be merged.

In this description of an example flow of a method 700 for recording and modeling electrical activity (particularly cardiac electrical activity) of a patient, the description follows, first, the straight-line process, then returns to decision boxes (broken diamond shapes) to fill out the description of example alternative flows. The method represents a computer process that records and processes biopotential data, or other cardiac or biometric data, and generates one or more outputs, including display and/or electronic signals and transmissions.

In this example embodiment the process begins in step 702, where system 5000 acquires cardiac biopotential data over one or more cardiac cycles. As described in the discussion related to FIG. 2, the data may be recorded when the recording elements are, or are not, in contact with cardiac tissue, or when there exists a combination of contacting and non-contacting recording elements, e.g., electrodes. Other data acquisition options are described in greater detail with reference to FIG. 2.

After acquiring biopotential data in step 702, the process moves to step 704, during which the biopotential data is filtered. As described in greater detail in the discussion related to FIG. 2, system 5000 may employ any of a variety of filtering techniques, such as a V-wave filter, as an example.

In step 706 cardiac cycles (e.g. beats) are identified and cycle lengths (CL) are calculated. After calculating cycle lengths, in step 708 system 5000 determines whether the calculated cycle length is consistent with any current beat group. If the cycle length falls within a threshold value of a current beat group, the process proceeds to step 710, during which cardiac biopotential data is evaluated based on secondary characteristics, such as timing pattern or signal morphology/shape on one or more recording elements, such as one or more of the electrodes described herein.

Then, in step 712, system 5000 may employ a data clustering method to determine whether secondary characteristics are matched to an existing beat group, for example. In step 714, biopotential data may be segmented on the basis of beat cycle length and, in step 716, the system may filter, or exclude, unwanted data from the set of acquired beat biopotential data for each channel/electrode. Unwanted, and discarded, data may include noise, artifacts, erroneous measurements, or disconnections, for example. Such unwanted data can be detected and filtered out and discarded using any of a variety of filtering techniques.

After filtering in step 716, system 5000 may assign a current beat to a beat group in step 718, and proceed from there to step 720, during which system 5000 determines whether to update the cardiac map data “on-the-fly,” e.g., in real or near real time, and then to step 722, during which system 5000 determines whether or not to continue acquiring biopotential data.

From step 722, the process proceeds to step 724 during which system 5000 time-aligns all the beats within a beat group to create an aggregated/accumulated measurement set for a plurality of locations of the heart over a single cardiac cycle. In step 726, system 5000 models cardiac electrical activity based upon the aggregated/accumulated measurement set.

Returning to steps 708 and 712, the system may initiate a new beat group in step 728 if the beat's cycle-length is not consistent with a current beat group (decision box 708) or secondary characteristics do not match a current beat group (decision box 712). From step 728, the process proceeds to step 730, during which biopotential data is segmented based on the beat's cycle length, then to step 732, during which unwanted data is filtered from the set of beat biopotential data. The process then proceeds to step 734, during which system 5000 creates a model of characteristics that future beats may be “matched” to. Then the method proceeds to step 718, during which the process proceeds as previously described.

Returning to step 720, if system 5000 updates the cardiac map on-the-fly, then the process proceeds to step 736, during which system 5000 time-aligns all beats within a beat group to create an aggregated/accumulated measurement set from a plurality of locations over a single cardiac cycle. The process then proceeds to step 738, during which the system models cardiac electrical activity based on the aggregated/accumulated measurement set and, from there, to step 748 to display cardiac electrical activity and, optionally, and any related data and/or information. The process can return to step 702, such that it can continue to acquire biopotential and location data, as previously described and then further process and display cardiac activity and any related data and/or information.

Returning to step 722, the process returns to step 702 during which it continues to acquire biopotential and location data, as previously described, should the system determine to continue acquiring data.

From step 702 the system may proceed to step 740, during which it models cardiac electrical activity based upon an aggregated/accumulated measurement set, when sufficient data has been acquired. This step can be in preparation for generating a cardiac activity display, or for further processing of electrical cardiac data, or for both.

From step 712, or from step 740, the system may proceed to step 742, during which the system can generate one or more displays of a quality and distribution summary of all previous acquisition locations of beats assigned to the current beat group. From step 742, the process proceeds to step 744, during which the system updates the display of quality and distribution summary to include the current beat. In step 746, the system can temporarily display one or more locations of new data added to the beat group by highlighting the associated electrode, using, for example, flashing of electrode color, displaying rings around the electrodes, sounding a tone, displaying a callout, or other highlighting or distinguishing methods. In step 748, the system displays the cardiac electrical activity, which can include a graphical display of at least one chamber of the heart with cardiac activity data superimposed thereon.

From step 718, the system may proceed to step 744, during which the system updates the display of quality and distribution summary to include the current beat, and, from step 744, as previously described.

From steps 726 and 738, the process may proceed to step 748, to display cardiac electrical activity.

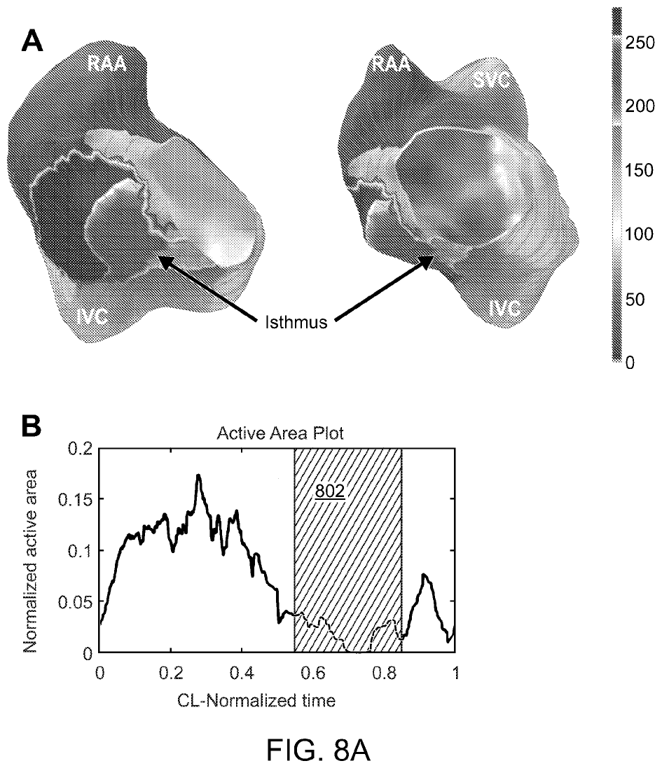

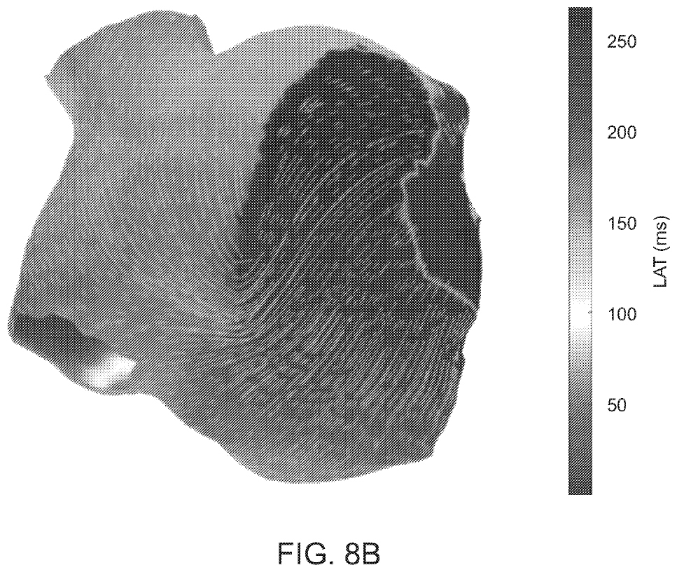

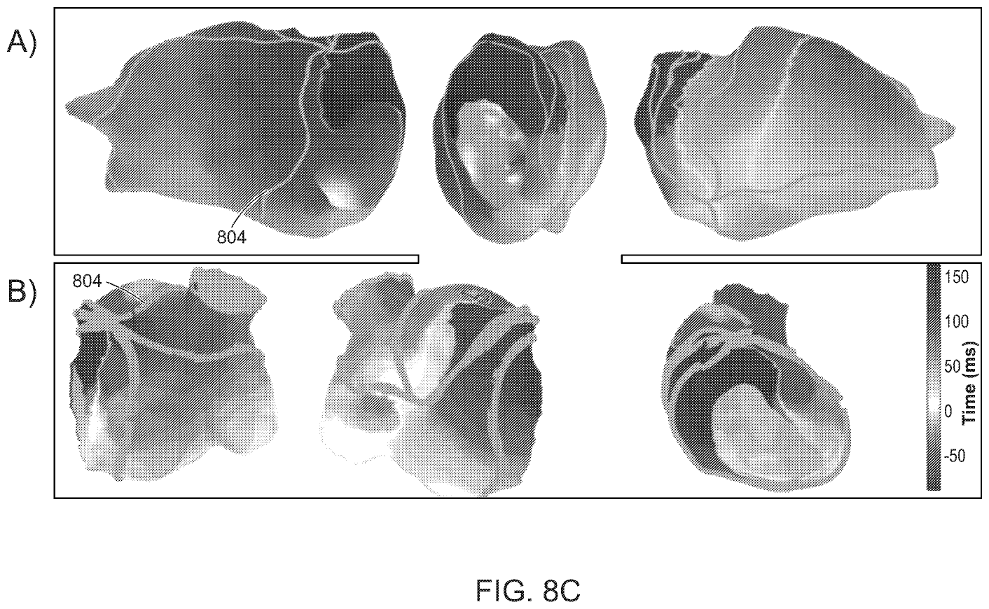

Referring now to FIGS. 8A-C, various displays of cardiac activity maps are illustrated, consistent with the present inventive concepts. The maps and displays can be generated by system 5000 of FIG. 1.

In particular, FIGS. 8A through 8C illustrate conduction isthmus visualization that may be provided by a system and method in accordance with principles of inventive concepts, for example, to aid a physician in a cardiac ablation procedure to treat or terminate an arrhythmia. Complex atrial tachycardias (AT) can be challenging to map and ablate, at least in part, because ablation-induced scarring can result in low amplitude electrical activity, and, therefore, make it more difficult to detect and map atrial activity in and around ablation-induced scarring. Because the regions of low amplitude activity often contain the site (critical isthmus) where ablation is used to terminate the arrhythmia, it is critically important to provide the physician with a clear, detailed, view of the activity, despite the diminished, low-amplitude, signal levels. In example embodiments, a system in accordance with principles of inventive concepts may employ any one of, or any combination of, active area plots, streamline plots, or AutPath plots.

Active Area Plots

An active area (AA) plot may be used to help identify the conducting isthmus and may be constructed by plotting the normalized cycle length of a cardiac rhythm on an X axis and the normalized amount of depolarized tissue on a Y axis. For each portion of the rhythm cycle length, the percentage of atrial tissue depolarized may be determined by counting all vertices of an anatomy with local activation times around a time point within a rhythm cycle length (within +/− a time window, such as 20 ms, for example) and dividing by the total number of vertices of the anatomy. The vertices referred to may be those of polygonal shaped projections onto the epicardial surface forming a mesh, wherein system 10 computes the dipole density at all vertices. Such polygonal shaped projections can be in the form, in some embodiments, of a series of abutting triangles forming a mesh on the cardiac surface. For example, for a rhythm with a cycle length of 300 ms, a time point 0, all vertices with activation within 0-20 ms to 0+20 ms can be counted and divided by the total number of vertices. Vertices that are active during the valleys in AA maps are often concurrent with isthmus conduction.

FIG. 8A shows an example of an AA plot. In example embodiments, computed activation times from electrograms are directly used in computing the depolarized tissue area, employing charge density or unipolar voltage directly. The method shown here is similar to the method described in Automatic Identification of Reentry Mechanisms and Critical Sites during Atrial Tachycardia by Analyzing Areas of Activity, IEEE Trans. Biomed. Eng., no. February, 2018, T. G. Oesterlein, A. Loewe, G. Lenis, A. Luik, C. Schmitt, and O. Doessel, which is hereby incorporated by reference. However, a key difference is that in Oesterlein et al., the authors threshold a function based on the raw electrograms to determine the amount of tissue depolarized, whereas in this approach, the computed activation times from the electrograms are directly used in computing the depolarized area. Additionally, the approach in Oesterlein et al. uses filtered bipole electrograms for the determination of depolarized area whereas this approach uses unipolar voltage directly or charge density.

In the lower half of FIG. 8A, labeled B, an AA plot shows the amount of depolarization tissue at each moment during the AT cycle length. A shaded vertical band overlay 802 highlights the valley in the AA plot. In the upper half of the figure, labeled A, is a local activation time (LAT) map with a stream line (SL) indicating the flow of conduction. The highlighted area in the 3D map corresponds to the valley in the AA plot, shown in the shaded overlay. SLs begin outward type conduction after leaving isthmus type conduction. In this example, the patient had a gap in a previous CTI line, which corresponds to the highlighted region in the map.

Streamline Plots

Streamline plots are a method in accordance with principles of inventive concepts to help visualize the flow of conduction; regions of streamline converging can indicate isthmus conduction. Streamlines show the path that conduction takes on the anatomy (as shown in FIG. 8B). Given a vector field, \vec{u}, streamlines, \vec{xₛ}, can be defined as:

$$\frac{d \vec{{x}_{s}}}{ds} x \vec{u} ( \vec{{x}_{s}} ) = 0$$

This definition indicates that the streamlines run perpendicular to vectors in a vector field. Streamlines can be constructed for cardiac conduction by defining the vector field, \vec{u}, as the unit vector conduction velocity field for a rhythm. With \vec{u} defined, a streamline \vec{xₛ} can be approximated given an arbitrary point on the streamline, \vec{xₛ_{ₜ}}. From the starting point, \vec{xₛ_{ₗ}}, a forward step along the streamline, \vec{xₛ_{ₗ₊₁}}can be found by taking a small step in the positive \vec{u}(xₛ). This step is taken as:

\vec{xₛ_{ₗ₊₁}}=\vec{xₛ_{ₗ}}+\vec{u}(xₛ)*Δt

Likewise, a backward step along the streamline can be computed by taking a small step in the negative \vec{u}(xₛ). This step is taken as

{right arrow over (xₛ_{_{l−1}})}=\vec{xₛ_{ₗ}}−\vec{u}(xₛ)*Δt

This forward and background propagation continues until \vec{xₛ} reaches either: 1) a line of block, defined as a large time difference between consecutive steps along the streamline or a rapid change in path direction along the streamline (e.g. direction change greater than 90 degrees) or 2) the streamline passes across the early meets late activation timing. A collection of streamlines can be created from an arbitrary density of seed points. FIG. 8A, top, indicates streamlines shown in grey overlaid over the LAT map.

Once found, the streamlines can be animated on the anatomy to indicate conduction flow. The animation can be set by initializing time t and then progressing each streamline individually with a vector or particle, as illustrated in FIG. 8B. In example embodiments, animated objects along the streamline can have the objects change along the streamline to indicate certain properties of the anatomy/substrate. For example, the speed of the animated object may change along regions of slower conduction, or the size of the object may change to indicate the relative electrical mass of the tissue (amplitude). Arrows, lines, particles, or other objects may be animated to progress along the streamlines. Animating the streamlines gives the appearance of flow over the anatomy, enhancing the visualization for a physician.

Auto Path

Auto path is a method in example embodiments in accordance with principles of inventive concepts to automatically indicate on a cardiac anatomy the likely pathways for reentry, given the patient's LAT map (or propagation history). Reentry can be defined as an abnormal electrical impulse that continually self-sustains, instead of dying out, dissipating, or collapsing by colliding with unexcitable tissue. Reentry may be visibly identified by finding a pathway of electrical activity (usually in the form of LAT times) that forms a closed loop. For example, if a reentrant arrythmia has a cycle length of 300 ms, any pathway that can be traced from Oms to 300 ms then back to 0 ms, while maintaining normal physiological conduction properties (for example, minimum and maximum conduction velocities) may be considered a possible reentrant pathway.

Shortest-path algorithms may be used to find candidate reentrant pathways and those candidate pathways may then be further evaluated to determine whether the pathways maintain normal physiological conduction. Given an anatomical mesh and LATs on the mesh, edge weights may be computed to indicate the cost of traveling from one vertex to another. Edges from one vertex to another that follow physiological conduction given the LATs on the vertices can be given low weights, whereas less physiological or certain conduction from one vertex to another can be given higher weights.

Candidate pathways between two vertices can be found by minimizing the sum of edgeweights to travel between the two vertices because more physiological conduction will have lower edgeweights. A plurality of algorithms to efficiently find pathways that minimize the cost for traveling between paths are known and may include using Dijkstra's algorithm. The edgeweights between adjacent vertices may be constructed based on the angle between the conduction velocity and the distance vector between the two adjacent nodes, as well as the LAT time difference between the adjacent nodes. One such formation could be:

$$edgeweight ( {v}_{1} , {v}_{2} ) = α ( 1 - dot ( \frac{\vec{{CV}_{{v}_{1}}}}{‖ \vec{{CV}_{{v}_{1}}} ‖} , \frac{\vec{X ( {v}_{2} ) - X ( {v}_{1} )}}{\vec{‖ X ( {v}_{2} ) - X ( {v}_{1} ) ‖}} ) ) + ( \vert {LAT}_{{v}_{1}} - {LAT}_{{v}_{1}} \vert ) ) , \qquad ( Equation 1 )$$

Where v₁, v₂ are two adjacent vertices, \vec{CVₕ_{ₙ}}, is the conduction velocity at vertex, n, X(vₙ), is the position of the vertex, n, LATᵥ_{ₙ} is the LAT time of vertex, n, and a is a scaling constant to set the relative importance of LAT time difference and difference in conduction velocity direction and edge direction.

The algorithmic sequence for automatically determining possible reentrant pathways on a cardiac anatomy given a LAT or propagation history map may be summarized as follows:

Algorithm—To automatically find reentrant pathways:

Input: Number of seeded reentrance pathways, r (usually 80-1000), mesh (vertices and faces), LATs on mesh.
a. Construct edge weight matrix using the anatomical mesh and LATs on the mesh
b. For every r:
   i. Randomize two vertices on the mesh, v1, v2
   ii. Find path, p1, to minimize edge weights between v1 to v2
   iii. Find path, p2, to minimize edge weights between v2 to v1
   iv. Concatenate p1 and p2 to form candidate path, candidatePathₙ
c. For every candidatePathₙ
   i. Remove candidatePaths that do not meet some conditions (e.g., has a minimum path length, contains minimum cycle length, maintains conduction velocities within physiological ranges) to form a set of confirmedReentrantPaths.
   ii. Cluster/Group confirmedReentrantPaths that are similar based on features such as frechet distance, other features may be used, such as shape descriptors, in combination with unsupervised learning techniques.
   iii. Determine a representative confirmedReentrantPath from each of the Cluster/Groups found above.
   iv. Filter/smooth representative confirmedReentrantPath. Filtering may include mean/median filtering, active snakes/contouring filtering, and/or other filtering-based techniques.

Once the pathways are found, one or more of the pathways can be displayed on, or in conjunction with, the anatomy.

FIG. 8C illustrates the example output of an automatic path annotation algorithm on two cases overlaid on LAT maps, in accordance with principles of inventive concepts. Pathways of reentry are identified by grey lines 804 on the anatomy. At the top of FIG. 8C, labeled “A,” three potential pathways of reentry are identified automatically for a left atrial anatomy. At the bottom of FIG. 8C, labeled, “B,” three potential pathways of reentry are automatically identified in the right atrium.

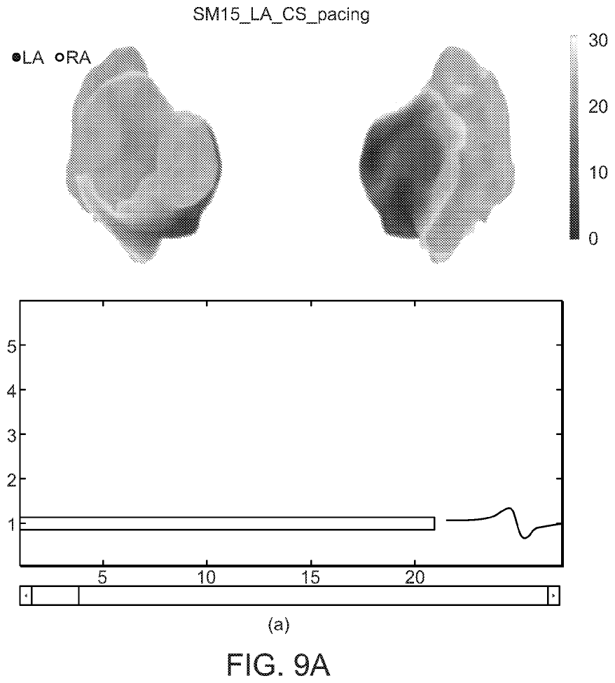

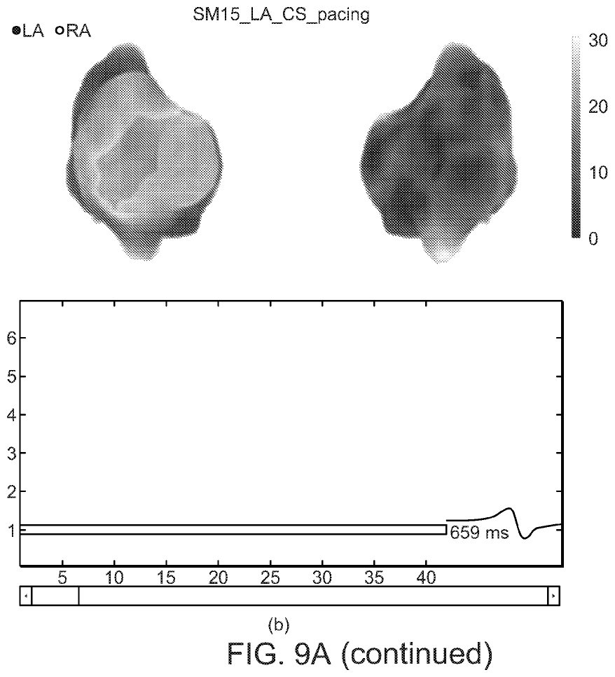

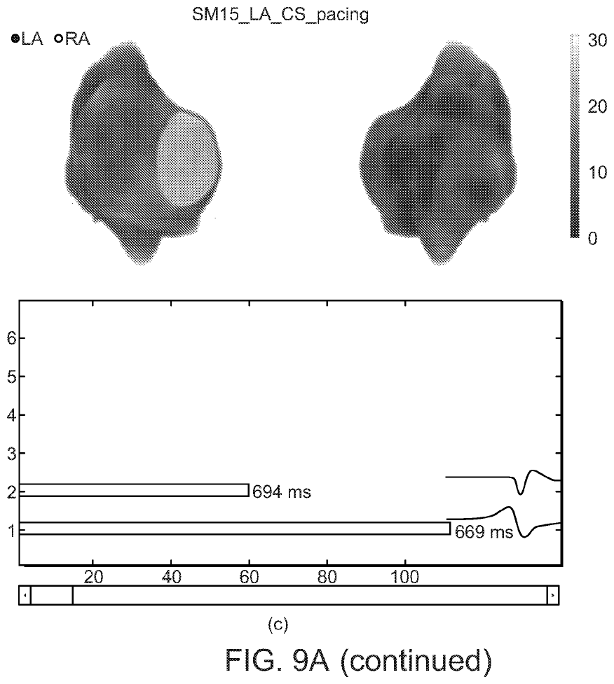

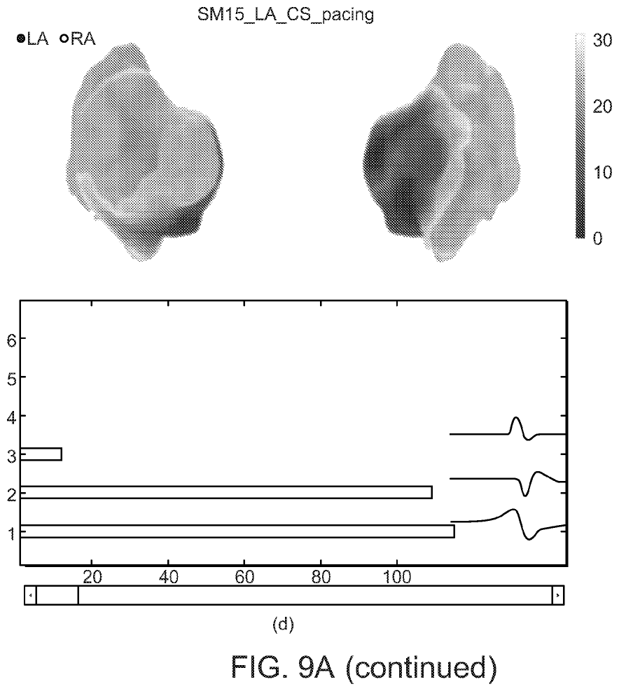

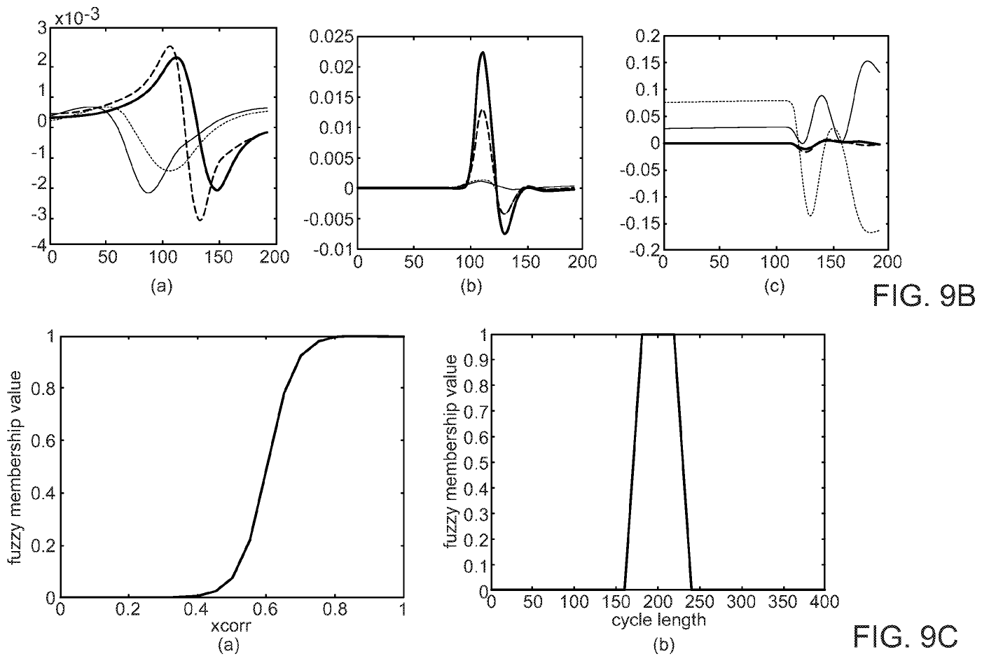

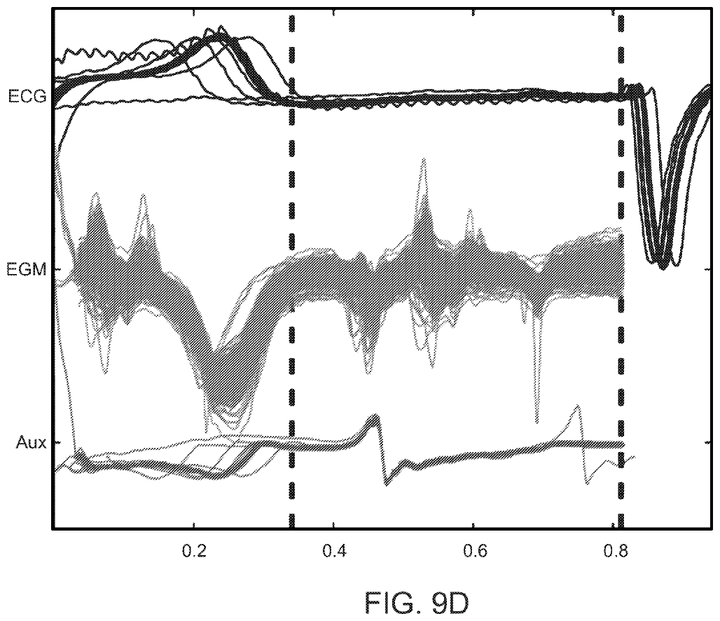

Referring now to FIGS. 9A-D, sequential data acquisition, cluster samples, fuzzy membership functions bio data for beats within a given group are illustrated, consistent with the present inventive concepts. In example embodiments, real-time clustering may be employed to assist a physician in visualizing cardiac structure and activity. Bio data (such as CS or ECG) may be grouped in real or near real time. That is, each incoming beat can be assigned to an existing group or to a new group. Such real-time clustering may be achieved in systems and methods in accordance with principles of inventive concepts, as follows:
a. Buffer processing: the steps described here are similar to the steps in offline clustering.
   i. The initial steps are to get activation on CS channels, while omitting activation on QRS (QRS blanking), and remove CS and ECG channels that have artifacts.
   ii. Segment the CS channels around the activation. This segment is usually smaller than a cycle length. It usually covers only “active CS time” (narrow band segmentation).
   iii. Cluster the segments using current wavelet-based clustering method and pick a representative sample from each group as a mean template. The representative sample can be selected as the sample which has the highest cross correlation to the mean sample of that group.
b. Loop until sufficient data acquired
   i. Get new bio data (can be set fix or variable length. For example, it can be set as greater than 1.2*minimum cycle length of existing groups)
   ii. On new bio data, detect activation on CS channels, estimate QRS width and cycle length
   iii. Grouping/clustering
      1. For the new beat, check if cross correlation to the template of any group is greater than a threshold, then assign the group label to which the new beat has the highest cross correlation. (cross correlation can be normalized 2D cross correlation, CS channels X samples) or statistical metric such as mean, minimum or maximum of 1D cross correlation of each channel.
      2. If the cross correlation is lower than threshold, perform wavelet decomposition and keep k coefficients. The k coefficients can be the k largest valued coefficients, or they are selected from fixed indices.
         a. Create superclusters using a hierarchical clustering method on wavelet coefficients.
         b. If any supercluster has sufficient # of samples, assign a new group label to the samples, and mark a representative sample as a mean template.
      3. Add 2 cycle length of basket potential data to the corresponding group's basket potential data, while removing bad electrodes (high amplitude, outside, motion)
c. Clear basket potential data further by Gaussian mixture model (artifacts, high amplitude)

In example embodiments, the methods mentioned in the clustering step (b)(iii) may also performed by other methods. For example, cross correlation-based matching can be replaced by wavelet transform based matching. Selecting k coefficients in wavelet transform can be replaced by dimensional reduction method such as approximated T-distributed Stochastic Neighbor Embedding (TSNE), principal component analysis.

In example embodiments, during the acquisition visual feedback may also be provided for each cluster. The visual feedback can show for each cluster heart chamber coverage (distance to the closest electrode), number of beats, CS signals and cycle length. An example of visual feedback is shown in FIG. 9A, which illustrates sequential data acquisition for sinus, CS proximal and distal pacing. Each subfigure displays coverage on anatomy for a selected cluster and a horizontal bar plot showing the number of clusters and number of beats for each cluster. Along the bar plot, reference CS channel for each cluster is also shown. The subfigures are as follows: subfigure (a) After buffering step; subfigure (b) During Sinus rhythm acquisition; subfigure (c) proximal CS pacing (coverage is for cluster 1); subfigure (d) distal CS pacing (coverage is for cluster 3).

FIG. 9B illustrates the corresponding CS channels, with representative sample from each cluster. The subfigures are as follows: subfigure (a) sinus rhythm, subfigure (b) proximal CS pacing, and subfigure (c) distal CS pacing.

In the above grouping algorithm, only morphology characteristic is considered. In accordance with principles of inventive concepts, other characteristics, such as cycle length, activation time sequence/pattern may also be considered. Multiple characteristics can be applied in a sequential manner, as described in the discussion related to FIG. 7, above, or may be combined in a single step using fuzzy membership functions.

Fuzzy membership functions convert any real-valued function to probabilistic valued function (1=pass, 0=fail, other value=fuzzy(uncertain) zone). For multiple characteristics, the total cost in fuzzy membership function can be written as:

F(λ)=λ_{1 μCL}+λ_{2 μmorpth}+λ_{3 μActTime},

where λ₁+λ₂+λ₃=1, {λ₁, λ₂, λ₃}∈[0 1], and λ={λ₁, λ₂, λ₃} control the influence of each membership function(μ).

In example embodiments, cycle length and morphology can be combined as follows for a given specification.

In various embodiments, a specification can be: (a) mean cycle length is 200 ms, allowable cycle length change is 10%, and more than 20% change in cycle length is considered as a different cluster; (b) cross correlation threshold is 0.8, and 50% confident to belongs to the same cluster, when cross correlation value is 0.6. For these specifications, the fuzzy membership functions can be calculated as shown in FIG. 9C. Now, for each incoming beat, total cost F(λ)=λ1 μCL+λ2 μmorpth is calculated for given values of λ and then check if the cost value is above threshold or not. FIG. 9C illustrates fuzzy membership functions as follows: subfigure (a) for cross correlation (μmorpth), and subfigure (b) cycle length F(λ)=(μCL).

Auto Segmentation

For a given group, one cycle length data of EGM data can be segmented automatically as follows. For each beat of a given group, two cycle length of EGM, ECG, and CS data are collected and stacked together. The data is collected as one cycle length before and after for each reference CS channel activation (see FIG. 9D). Within the two-cycle length of data, automatic segmentation provides the two end points that are one cycle length apart such that within that one cycle length data, the number of beats having QRS and T-waves are as low as possible (see black dotted lines in FIG. 9D).

In FIG. 9D, two-cycle length of bio data for all beats within a given group are stacked together. The black dotted lines are estimated by automatic segmentation method. The lines are one cycle-length apart and the segments within it have QRS and T-wave as low as possible.

In addition to above constraints, in example embodiments, additional constraints may be added. For example, a constraint that the end points should have either low or high EGM (envelope) energy value. This constraint may be implemented by the following steps:
a. Estimate EGM envelope and calculate energy (square of voltage)
b. Estimate Onset and offset for T-wave and QRS for each beat.
c. Find potential end points where EGM energy is low.
d. For each possible end point pairs, calculate the cost—how many beats have T-wave and QRS and what percentage of the cycle length they cover. The cost calculation can be carried out with different weight for QRS and T-wave.
e. Pick the end points that has the lowest cost.
f (Optional) Throw away segments that have non-zero cost if we have enough beats having zero cost.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for modeling a patient's cardiac electrical activity data, comprising:
at least one diagnostic catheter for insertion into the heart of the patient, the at least one diagnostic catheter comprising at least one recording element configured to record patient data over multiple cardiac cycles, the patient data comprising:
biopotential data; and
stored and/or received localization data comprising the location of the at least one recording element; and
a processing unit comprising a clustering routine configured to:
receive the recorded patient data;
segment the recorded patient data by cardiac cycle to produce segmented patient data comprising segments;
group the segments based on one or more characteristics of the segments to produce segmented data groups; and
combine the segmented patient data within each segmented data group to produce one or more composite recordings;
wherein the system is configured to create one or more models of cardiac electrical activity of the patient based on the one or more composite recordings.

2.-55. (canceled)
